(19) United States
(12) Patent Application Publication
(10) Pub. No.: US 2023/0192548 A1
(43) Pub. Date: Jun. 22, 2023

(54) ENZYMATIC CONSTRUCTION MATERIAL FOR REPAIR AND CORROSION RESISTANCE AND DURABILITY

(71) Applicant: Worcester Polytechnic Institute, Worcester, MA (US)

(72) Inventors: Shuai Wang, Worcester, MA (US); Nima Rahbar, Weston, MA (US); Suzanne F. Scariata, Worcester, MA (US)

(21) Appl. No.: 18/110,484

(22) Filed: Feb. 16, 2023

Related U.S. Application Data
(63) Continuation-in-part of application No. 17/943,548, filed on Sep. 13, 2022.
(60) Provisional application No. 63/243,494, filed on Sep. 13, 2021.
(60) Provisional application No. 63/311,230, filed on Feb. 17, 2022.

(51) Int. Cl. C04B 24/14; C04B 41/63; C12N 11/14; C12N 9/88; E01C 7/14; E01C 7/35

(52) U.S. Cl. CPC C04B 24/14; C04B 41/63; C12N 11/14; C12N 9/88; C12Y 402/01001; E01C 7/147; E01C 7/35; C04B 2111/72

(57) ABSTRACT

Methods for repair of cracks and niches in concrete using carbonic anhydrase embedded in a gelatin, hydrogel, or other aqueous matrix are provided. A durable corrosion resistant concrete mix containing carbonic anhydrase is provided, as are methods of making and using the durable corrosion resistant concrete. Corrosion resistance and durability derive from decreased porosity compared to concrete without the enzyme, conferring decreased access of deleterious materials such as chloride ions to interior portions of the concrete.

### RELATED APPLICATIONS

This patent application is a Continuation-in-Part (CIP) under 35 U.S.C. § 120 of application Ser. No. 17/943,548 filed Sep. 13, 2022, which claims the benefit of U.S. provisional application No. 63/243,494, filed Sep. 13, 2021, each entitled “ENZYMATIC CONSTRUCTION MATERIAL”, each by inventors Nima Rahbar, Shuai Wang, and Suzanne Scarlata, and U.S. provisional application Ser. No. 63/311,230 filed Feb. 17, 2022, entitled, “Enzymatic repair of large concrete surfaces”, inventors Shuai Wang, Nima Rahbar, and Suzanne Scarlata, all of which are hereby incorporated herein in their entireties.

### TECHNICAL FIELD

Methods of making a cement construction material that with increased durability and corrosion-resistance, and methods of repairing defects in cement are provided.

### BACKGROUND

Concrete is an invaluable building material owing to its compressive strength and resistance to atmospheric elements. Concrete is a general term applied to a mixture of Portland cement and aggregate, typically sand and gravel of various sizes. The Portland cement reacts with water to bind the aggregate and cure to form a solid mass. Concrete is easily transported as a dry material having a powdery, gravel texture, and forms a fluidic, granular slurry type of material immediately after mixing with water. Prior to curing, the fluidic mixture may be cast or poured into any suitable shape; about 90 minutes after mixing with water it begins to cure into the molded shape and manifests substantial compressive strength and mass.

Apart from water, concrete is the most consumed material on earth as it has been calculated that three tons per year are used per person. Every year, waste concrete from construction and carbon dioxide emission from cement production and transport is increasing, and concrete alone contributes to 9% of total CO₂ emission. The average temperatures in the Arctic have been rising faster than anywhere on earth, this is due to the rise in sea level caused by the greenhouse effect. Climate change caused by increased CO₂ levels due to human activity is the biggest existential threat facing the world. Therefore, reducing carbon dioxide emissions to reduce the greenhouse effect is an urgent task. Methods to reduce concrete consumption with high-volume pozzolans replacements such as fly ash and slag in concrete consumption are not environmentally friendly.

Concrete in its many forms is one of the most widely used construction materials worldwide. A cumulative degradation of concrete is caused by salts, alkalis, freeze-thaw cycles, carbonation, and physical wear. Commonly used repair materials for concrete cracks mainly include epoxy systems and acrylic resins which are all environmentally unfriendly, and often cause delamination or cracking between the original concrete matrix and the repair material.

The repair processes for cracked and damaged concrete typically rely on matching dissimilar materials, such as inorganic calcium-silica-hydrate (C—S—H) compositions with organic petroleum-derived epoxies. However, patching and resurfacing are highly dependent on artisanal skill, and often cause further damage thereby undermining the repair process.

Given the enormous amount and worldwide extent of aging and decaying concrete in roads, bridges and buildings, there are urgent needs for materials and methods for repairing cracks and fissures in cementitious surfaces that are environmentally friendly and do not cause further damage, and for corrosion-resistant durable concrete and methods of making concrete mixes for construction materials that are characterized by greater ability to resist environmental degradation.

### SUMMARY

An aspect of the invention herein provides a method for repairing at least one fracture or notch in a cementitious surface, the method comprising:

treating at least one fracture or notch in the cementitious surface to at least one carbonic anhydrase preparation, the preparation containing carbonic anhydrase immobilized non-covalently on a particulate substrate or embedded in a semi-solid bead matrix; and subjecting the cementitious treated surface to ambient conditions of atmospheric carbon dioxide and temperature, thereby repairing the fracture. An embodiment of the method further includes repeating the treatment the at least one fracture or notch with the carbonic anhydrase preparation and subjecting the treated surface to the ambient condition. For repeating the treating step, the method further includes air drying the treated cementitious surface before repeating the treating.

In various alternative embodiments, the carbonic anhydrase preparation further includes at least one additional component selected from: a native carbonic anhydrase, calcium ions, a buffer, and water. The native carbonic anhydrase has an amino acid sequence found in nature, as this enzyme is found throughout the range of biological organisms, both in prokaryotes and eukaryotes, and the enzyme suitable for methods and materials herein may be obtained from any source. The carbonic anhydrase preparation particulate substrate is in various alternative embodiments one or more of a silicone bead, silicon dioxide, a silica gel, disiloxane, silicic acid, silanol, an organic silicon compound, sand, grit, cellulose, and a cellulose derivative. The carbonic anhydrase preparation semi-solid bead matrix is in various embodiments prepared from at least one selected from: a hydrogel, an agar, and a gelatin.

In an embodiment of the method, the CA is recombinantly produced. Alternatively, the CA is a purified or partially purified from a food industry byproduct or a fermentation byproduct. The carbonic anhydrase is recombinantly produced or is prepared directly from a natural biological source, as widespread use of the enzyme will require bulk quantities, and the source will be dictated by economic factors. Carbonic anhydrase is found in organisms from bacteria to mammals. Economical and convenient sources of carbonic anhydrase are waste products of meat packing, for example, bovine blood, beer production, and other microbial fermentations that discard cellular materials.

The carbonic anhydrase in various embodiments is purified or is semi-purified or is a crude extract, for example, produced from a crude lysate of cells by organic solvent extraction or by a small number of ammonium sulfate precipitations. Thus in various alternative embodiments the carbonic anhydrase is purified or partially purified from a biological source, for example, is a mammalian enzyme byproduct of the meat industry produced, for example, from bovine blood or is a bacterial enzyme or yeast enzyme byproduct of antibiotic or high-value protein fermentation. Certain applications of the method may involve obtaining carbonic anhydrase from a bacterial species that is a thermophilic or a xerophilic species. The recombinant carbonic anhydrase may be produced by expression of the gene encoding the enzyme, expressed in E. coli, in Streptomyces lividans, or in Saccharomyces cerevisiae, for example, secreted into the fermentation medium.

Another aspect of the invention herein provides a method for improving durability and corrosion resistance of a cementitious surface, the method including steps of:

contacting a cement mixture prior to curing to at least one carbonic anhydrase (CA) preparation to form an enzyme-cement mixture (ECM);

applying the ECM to a surface and subjecting the ECM on the surface to an ambient atmosphere and curing, thereby improving durability and corrosion resistance by reducing porosity of a resulting CA-contacted cementitious surface in comparison to a control cement not CA-contacted.

The method uses the carbonic anhydrase preparation with the enzyme embedded or immobilized non-covalently on a particulate substrate selected from a silicone bead, a silica gel, sand, grit, cellulose, cellulose derivative; or the carbonic anhydrase preparation comprises CA embedded in a semi-solid bead matrix comprising a hydrogel or in gelatin. Embedding or immobilizing or attaching the CA provides better distribution in the cement mixture in comparison to a CA solution. While gelatin has a low transition temperature from solid or semi-solid to liquid, association of the enzyme even with gelatin in a liquid form is beneficial to the distribution of enzyme in the resulting cementitious product. Matrix materials with higher melting points such as agar can be used in low percent preparations, for example less than 1% or 2% agar, and agar/gelatin mixtures are also envisioned.

The ECM materials herein and methods of making the ECM provide for the resulting CA-contacted cementitious surface having reduced permeability to deleterious environmental corrosive agents. The reduced porosity results from calciferous crystallization shown herein to be due to the action of the carbonic anhydrase. An important consequence of reducing porosity of the resulting cementitious surfaces is decreased permeation by deleterious salt ions including chloride salts. Corrosion by chloride ions of iron rebars in reinforced concrete used in construction is thereby prevented or reduced.

In various embodiments of the methods herein, the contacting step further includes adding Ca⁺⁺ ions to the ECM. The method operates without adding Ca⁺⁺ under circumstances in which environmental Ca⁺⁺ is sufficient as calcium is the fifth most common element in earth's crust, and the step of adding Ca⁺⁺ ions speeds up the resulting process of reducing porosity. In an alternative to adding calcium to the ECM, the applying step includes adding Ca⁺⁺ ions to the surface after the contacting step, for example before or after curing. For example, the Ca⁺⁺ is a solution of calcium formate.

An aspect of the invention herein provides a cement alternative known as ECM having improved durability and corrosion-resistance, the cement mix comprising a carbonic anhydrase enzyme in an aqueous solution, the enzyme attached or embedded or enmeshed in a particulate admixture, the enzyme associated with at least one matrix selected from gelatin, agar or a hydrogel, or associated with at least one particulate material selected from silicone beads, silica gel, sand, grit, cellulose and cellulose derivatives, at a pH of 4.5-9.5.

The corrosion-resistant cement mix in various alternative embodiments contains the enzyme at a concentration in a range selected from the group of 100 nM to 500 nM, 500 nM to 1 μM, 1 μM to 5 μM, and 5 μM to 10 μM. An embodiment of the cement mix further includes Ca⁺⁺.

In a cement mix containing gelatin, compositions herein and methods of making and using these compositions provide concretes having corrosion resistance and durability and methods of repairing cracks in concrete. Additional examples are found in Matter 5: 1-18 published Mar. 2, 2022, authors Wang, Shuai, Scarlata, Suzanne F. and Rahbar, Nima, and which is incorporated herein in its entirety by reference.

### BRIEF DESCRIPTION OF DRAWINGS

FIG. 1 is a schematic drawing illustrating the preparation and fabrication of enzymatic construction material (ECM), and the enzymatic healing process.

FIG. 2 is a graph showing the weight gain for four different material designs with constant CO₂ gas treatment with five bubbles per second rate. Adding a base increases the weight gain and the weight gain is the highest for ECa samples. In the box plot, the median value is illustrated as (-), the mean as (□), the values at 25% and 75% as box edges, and the values at 5% and 95% level as whiskers. The weight gain is observed to be due fixation of the CO₂ by the action of the carbonic anhydrase.

FIG. 3 is a set of microphotographs showing the scanning electron microscope (SEM) images and energy dispersive spectroscopy (EDS) maps comparing the effects of the CA enzyme, calcium source, and high pH as aqueous solutions additives on different samples. The catalysis of the CA enzymes results in an ECM with an organized structure. Calcium is distributed uniformly on the ECM surfaces and bridges. Compared to control GL (Gelatin), GCa (Gelatin+Ca²⁺), and BGE (Base+gelatin+CA+Ca²⁺) samples, the mineral bridges in the ECM have wider and longer dimensions. The EDS maps of the ECM samples also show the highest amount of calcite crystals in comparison with the other three samples. The calcite bridges and organized structure distributed throughout the ECM provide greater strength, durability, and decreased porosity.

FIG. 4A-FIG. 4F is a set of microphotographs. FIG. 4A and FIG. 4B show the dimension of a typical crystal bridge in the ‘non-gelatin’ sample, ECa, is smaller compared to a typical bridge in ECM. These data are evidence that gelatin can establish a proper scaffolding framework for crystal formation that further enhances and extends the size of the crystal bridges. FIG. 4C and FIG. 4D show that a typical crack initiates and grows in the calcite bridges bonding the sand particles. The properties of the enzyme in gelatin are harnessed herein to provide methods of repair of cracks and fissures in existing concrete. FIG. 4E and FIG. 4F show detachment of the sand particles from the matrix.

FIG. 5A is a photograph of a CA sand slurry sample prepared with #No. 50 (the sand retained on a 300 m sieve) white sand. FIG. 5B is a photograph of the CA sand slurry sample after the uniaxial compression test. FIG. 5C is a photograph of a CA sand slurry sample prepared with #No. 30 coarse sand (retained on a 600 m sieve). FIG. 5D and FIG. 5E are a set of graphs showing the compressive strength and tangent modulus respectively of the fine sand slurry samples prepared with five different aqueous solutions. ECM is observed to show the highest compressive strength (9 MPa) and shows the highest tangent modulus.

FIG. 6A is a photograph of single-edge notch bending (SENB) test. FIG. 6B and FIG. 6C are a set of graphs showing the fracture energy, and the load-displacement curve respectively of three different groups of sand slurry beam samples: GL, GCa and ECM. The results show that ECM samples have the highest fracture energy and ultimate load, leading to a product with greater durability.

FIG. 7 is a set of microphotographs showing optical (top row), and SEM (bottom row) images of the process of enzymatically catalyzed mineral precipitation and growth on the sand-gelatin system within the first 20 minutes of curing.

FIG. 8 is a graph showing the time-dependent mineral thickness ratio (H=R) as a function of time. H is the thickness of a mineral bridge, and R is the average thickness of the gelatin network for sand-gelatin system. The solid line represents the numerical result from the diffusion model, and the dots present the data.

FIG. 9A and FIG. 9B are a set of photographs of a single-edge notch ECM beam samples before the fracture test and after the fracture test respectively. FIG. 9C is a photograph that shows that the fractured samples were placed in the silicon molds and treated with (3 parts) calcium-enzyme solution and CO₂ gas along the crack region. FIG. 9D is a photograph that shows that after treatment, the crack is healed. FIG. 9E is a set of photographs that show the healed samples after the desiccation in which the calcium carbonate crystals are visible in the fractured region. The specimen was re-healed in every cycle and the ultimate fracture load for every six cycles is indicated in FIG. 9F.

FIG. 10 is a schematic drawing of the self-healing mechanism in which after the fracture, upon the application of CO₂, CA's enzymatic catalysis reestablishes the broken mineral bridges using calcium and reconstructs the bridging network that holds the materials' microstructure together.

FIG. 11 is a graph of the compressive strength of ECM (n=14), glutaraldehyde-modified ECM (n=7), and tannin-modified ECM (n=5). Glutaraldehyde-modified ECM samples exhibit an impressive 12 MPa strength on average.

FIG. 12 is a powder X-ray diffraction (pXRD) analysis of the crystals produced by the CA enzyme method compared to the reference spectrum for calcite. The comparison confirms the crystallinity of the product resulting from the enzyme method is calcite.

FIG. 13 is a set of graphs showing Fourier Transform Infrared Spectroscopy (FT-IR) analysis of the enzyme-produced Ca-carbonate and compared to a reference spectrum in which the sample precipitate had a dry weight of 0.4 μg, confirming the chemical composition of the repaired product resulting from the enzyme solution.

FIG. 14A is a photograph of a coarse sand slurry cube. FIG. 14B is a graph showing the compressive strength of the coarse sand slurry cube with CA enzyme catalysis (Gelatin+Ca²⁺+CA) compared to intact control. The samples perform the highest average compressive strength 4.2 MPa.

FIG. 15A and FIG. 15B is a photograph and graph, respectively, showing fracture test for coarse sand slurry beams. FIG. 15C is a graph showing the result of coarse sand beam bending tests.

FIG. 16 is a set of microphotographs showing Optical microscopy images (top row) and SEM images (bottom row) of mineral precipitated and growth in a typical ECM sample within 10 minutes

FIG. 17A is a graph showing the concentration of calcium as a function of the radius of a bridge. FIG. 17B is a graph showing a linear approximation of the concentration profile.

FIG. 18 is a schematic drawing illustrating the preparation and fabrication of enzymatic construction material-n (ECM-n), the enzymatic healing process and the use of a laser for curing.

FIG. 19A is a photograph that shows the experimental setup of the laser-induced ECM-n curing. FIG. 19B is a thermal imaging showing the top view of the temperature variation of ECM-n. The maximum temperature rises to 81.5° C. at 60 minutes (n=5, Med=81.5, SD=3). FIG. 19C is a thermal imaging showing the side view of the temperature contours in ECM-n. FIG. 19D is a graph showing the temperature as a function of curing time at the center of a 0.1% nanoparticle-modified ECM-n 12.5 mm cubic sample surface under 3W laser induction compared with ECM without nanoparticles. Each data of ECM and ECM-n was taken from a median group of five independent samples (n=5).

FIG. 20 is a graph showing the normalized heat flow for the initial curing of ECM-n at 25° C. and 60° C. The behavior of the ECM-n heat flow after injecting an enzyme-calcite solution under two different temperatures. Both peaks can be identified within six minutes and then tend to plateau.

FIG. 21A-FIG. 21D are a set of Infrared thermal images of cured ECM-n thermal effect at different temperatures. FIG. 21A shows that the laser heating experiment was set up at ambient temperature for ECM-n. FIG. 21B shows the spatial view of the ECM-n at one-hour laser irradiation. FIG. 21C shows the side view of the sample at one hour of laser irradiation.

FIG. 21D shows the process of laser heating of ECM-n at −20° C. freezer and the sample's top surface temperature was raised to 60° C. in 10 minutes.

FIG. 22 is a graph showing the comparison of experiment and simulation of laser heating ECM-n. Temperature changes along the time and laser power for the ECM-n in 30 minutes. The laser was removed for twenty minutes and the materials started to cool to the ambient temperature. The results from experiments and the predictions show reasonable agreement.

FIG. 23A is a schematic illustration of the self-healing capability of the ECM-n beam via laser-induced heating. A trace amount of calcium-enzyme solution was added to the fractured area then ultra-pure CO₂ was aerated on the surface for 15 minutes. 3W laser was conducted in the same location over 4 hours. The heterogeneous shape of the high-temperature region (white color) develops into a circle gradually. FIG. 23B shows μ-CT scanned images of fracture and laser healing ECM-n beam at the front, center, and back layers.

FIG. 24A-FIG. 24I show a comparison of the multiscale microstructure and mineralization of ECM-n (left) and ECM (right). FIG. 24B-FIG. 24E show SEM and optical images of laser-cured ECM-n and oven-cured ECM showing the scaffold bridges and calcite crystals are distributed uniformly. FIG. 24F and FIG. 24G show higher magnification SEM images of scaffold bridges formed by laser curing and oven curing respectively. FIG. 25H and FIG. 24I show the EDS mapping images of the nanoparticle distribution in the matrix and the major chemical compositions.

FIG. 25A-FIG. 25B show the compressive strength of laser-repaired ECM-n with different flaws ECM-n samples with different flaw shapes that were repaired and compared with oven-cured ECM. FIG. 25A shows the laser repairing paradigm of ECM-n: The mean results are shown above each data set and a typical sample is shown at the bottom in FIG. 24B. The compressive strength of ECM, in order from left to right are: oven-cured ECM (n=12, blue, M=9.56, SD=1.49), laser-cured ECM-n (n=10, brown, M=9.45, SD=1.44), elliptical flaw ECM-n (n=10, grey, M=6.69, SD=1.92, P=0.003), circular flaw ECM-n (n=10, yellow, M=6.76, SD=1.79, P=0.004), repaired elliptical flaw ECM-n (n=10, light blue, M=9.76, SD=1.86, P=0.43) and repaired circular flaw ECM-n (n=10, green, M=9.47, SD=1.75, P=0.48).

FIG. 26A-FIG. 26C illustrate an Ashby diagram and scaling up ECM-n procedures Carbon footprint and mechanical properties of ECM/ECM-n compared to different construction materials. FIG. 26A shows an Ashby diagram of embodied CO₂ versus embodied energy data. FIG. 26B shows an Ashby diagram of specific strength versus embodied CO₂ for comparison with related construction materials. FIG. 26C shows the procedures of fabricating ECM-n on the construction site.

FIG. 27 shows photographs of incandescent light curing of the ECM-n sample. The ECM-n sample was cured in 12 hours at the ambient condition.

FIG. 28A-FIG. 28B show the comparison of mass loss and temperature over curing time between ECM n and ECM. FIG. 28A shows the variation of the sample's mass over time for ECM-n cured by 3W laser at room condition versus ECM. The data of ECM and ECM-n was taken from a median group of five independent samples (n=5). FIG. 28B shows the temperature as a function of fully cured time at the center of 12.5 mm ECM-n sample surface under 3W laser induction compared with EICM without nanoparticles. The data were presented by a median with a sample size of 3.

FIG. 29A-FIG. 29D show the thermomechanical modeling of ECM-n. The predicted three-dimensional temperature contour and the comparison of maximum temperature between experimental data and proposed prediction for ECM-n samples with different laser power. The heat transfer computation was carried out for three different powers. FIG. 29A shows the FEM mesh of ECM-n. FIG. 29B shows the Gaussian profile of order 1. FIG. 29C and FIG. 29D show the 3D and top views of the ECM-n showing the temperature contour at 20 minutes with a 3 W laser illumination.

FIG. 30A-FIG. 30B show the comparison of the self-healed ECM-n samples under the presence of carbonic anhydrase. FIG. 30A shows that fracture sample was healed by calcium solution and CO₂ without enzyme. FIG. 30B shows that the fracture sample was healed by calcium solution and CO₂ with the enzyme.

FIG. 31 shows the normalized Raman spectra of four different spots on the cured ECM-n surface. The peak analysis was processed and plotted by fitting the Gaussian function. The calcite and γFe2O3 positions were annotated in the diagram.

FIG. 32 shows a powder X-ray diffraction (pXRD) analysis of the enzyme-generated calcium carbonate in presence of laser induction, with a comparison to the reference spectrum for calcite. This comparison confirms the crystallinity of the product after laser curing resulting from the enzyme repair method.

FIG. 33 is a picture of a concrete slab that is 3 inches long and 3 inches wide having a fully repaired notch of 45 mm×4 mm×17 mm. The concrete slab was placed between two stands such that the notch was suspended. A gelatinous carbonic anhydrase enzyme was applied to the notch, the notch was air-dried, and the solution was reapplied to the notch. The process of applying the solution and airdrying was repeated for five days. The notch was observed to have been fully repaired in 7 days.

FIG. 34A-FIG. 34B are a set of pictures of a concrete slab that is 7 inches long and 4 inches wide having a fully repaired notch of 60 mm×5 mm×40 mm FIG. 34A shows that the length of the notch measured by a Vernier Caliper is about 56.4 mm. The concrete slab was placed between two stands such that the notch was suspended as depicted in FIG. 34B. A gelatinous carbonic anhydrase enzyme was applied to the notch, the notch was air-dried, and the solution was reapplied to the notch. The process of applying the solution and airdrying was repeated for five days. The notch was observed to be fully repaired in 14 days.

### DETAILED DESCRIPTION

A negative emission Enzymatic Construction Material (ECM) with self-healing capabilities can be used as an alternative to concrete and Portland cement. The disclosed approach employs carbonic anhydrase (CA) to catalyze the covalent condensation of carbon dioxide and water or the fixing of carbon dioxide from the atmosphere, to promote the precipitation of calcium ions in an aqueous solution as calcium carbonate crystals. As a result, a functional and biological ECM was obtained, whose compressive strength and Young's modulus properties are more than twice that of the cement mortars and other alternative building materials. The growth of mineral bridges that hold the sand particles were also modeled and studied. The approach provides a beneficial path for environmentally friendly construction materials.

Configurations herein depict a carbon-negative self-healing construction material compound, including a quantity of aggregates such as sand and gravel, a catalyst such as an enzyme, a scaffolding material having a crosslinking agent, and a calcium source.

The mixture including the enzyme is configured to bridge sand particles in the quantity of aggregates for forming a dense, solid mass. This enzyme-driven method to bridge the sand particles results in a dense, stiff, strong, and tough structural material, which upon exposure to calcium source and CO₂ can also heal itself repeatably. As described herein, the words catalyst and enzyme are interchangeably used.

Production of ECM adsorbs CO₂, hence not only it can reduce the current 9% global CO₂ emission caused by concrete production and repair, but it can also be used for carbon sequestration. The material is environmentally friendly, odorless, and harmless to humans and other organisms, with the highest mechanical strength (˜12 MPa) reported for an alternative construction material. Additionally, the curing process of ECM (a few days at ambient temperature) is significantly faster than traditional concrete at 28 days.

An aspect of the invention described herein provides a self-healing construction material compound, the compound including: an aggregate matter; a catalyst such as an enzyme; a scaffolding material; and a calcium source. In an embodiment of the compound, the compound is a carbon-negative compound.

In an embodiment of the compound, the aggregate matter further includes sand aggregates. In an embodiment of the compound, the scaffolding material further includes a crosslinking agent selected from glutaraldehyde, and tannin. In an embodiment of the compound, the scaffolding material includes a polymer. In an embodiment of the compound, the scaffolding material is gelatin. In an embodiment of the compound, the catalyst is an enzyme such as carbonic anhydrase or a chemical analog of carbonic anhydrase or a synthetically manufactured carbonic anhydrase.

An embodiment of the compound further includes a source of carbon dioxide. In an embodiment of the compound, the compound sequesters atmospheric carbon dioxide. The compound has a mechanical strength from at least 10 MPa to at least 16 MPa.

An embodiment of the compound further includes at least one of: a light source, a heat source, a laser source, and a magnetic field application source. In an embodiment of the compound, the compound is configured to form mineral bridges between the aggregate matter to obtain a dense mass. In an embodiment of the compound, the catalyst is configured to operate at a pH of 6.5 to 8.5 and to operate at a temperature of 30° C. to 50° C. An embodiment of the compound further includes a quantity of nanoparticles.

An aspect of the invention described herein provides a method for making a carbon-sequestering construction material, the method includes: preparing a solution having the catalyst and a calcium solution; mixing an aggregate matter with a scaffolding material to obtain a slurry; and adding the catalytic solution and the calcium solution to the slurry, such that the CA or analog utilizes carbon dioxide from the atmosphere, i.e., ambient carbon dioxide, and calcium from the calcium solution to form calcium carbonate crystals thereby sequestering carbon and obtaining a carbon-sequestering construction material.

In an embodiment of the method, the calcium carbonate crystals are deposited on the aggregate matter to create mineral bridges. An embodiment of the method further includes dehydrating the construction material for facilitating crosslinking between scaffolding material. In an embodiment of the method, the calcium solution is configured to facilitate continuous precipitation of the calcium carbonate crystals.

An aspect of the invention described herein provides a carbon-negative self-healing construction material compound, the compound including: a quantity of sand aggregates; a quantity of carbonic anhydrase catalyst; a gelatin scaffolding material; a calcium solution; and a quantity of iron oxide nanoparticles. An embodiment of the compound further includes a laser source or a light source for curing the compound.

Most of the infrastructure is made of concrete. Every ton of concrete produced releases one ton of CO₂ into the atmosphere. As a result, the concrete industry is the second largest industrial source of CO₂, accounting for about 9% of global emissions. The inventions described herein show a novel enzymatic construction material (ECM) with mechanical properties superior to concrete mortar. The fabrication method requires about 48 hours and is much faster than the fabrication method for current building materials including assemblies based on biological methods, which have a longer curing period and possess less than half of the mechanical properties of ECM.

Therefore, ECM is an alternative to cement that bridges the sand particles using a light and inexpensive polymer backbone through the deposition of calcium carbonate crystals, resulting in a hard, solid material that is twice as strong as the cementitious substrate and self-heals upon repeated large-scale cracking by incorporating an enzymatic catalyst. In contrast, cementitious materials or other current building materials do not have a similar capability. The state-of-the-art in active building material is living building material (LBM), which uses photosynthetic cyanobacterium to precipitate calcium carbonate crystals or fungi. LBM fabrication is inefficient because the cost of bacteria is much higher than ECM and the process cannot be not easily operated in large-scale manufacturing. Most important, the self-healing capability of the LBM is limited, and the material cannot regain its original strength. LBMs can also produce antibiotics such as polymyxin and refractory bacteria which can be biohazardous.

The inventions described herein are developed by applying biological methods to repair concrete through calcium carbonate deposition. Natural enzymes are proteins that catalyze a chemical reaction rapidly without being consumed in the process and are safe and reliable. Carbonic anhydrase (CA) is a fundamental enzyme found across all species. Fourteen isoenzymes of CA have been found in the human body. The structures, properties, and tissue distribution of the different CA isoenzymes are different; however, the isoenzymes have the same critical physiological functions in cell respiration by reacting CO₂ with water to yield carbonic acid in the body tissue and reversing the reaction in the lungs to generate CO₂.

The sequestration of CO₂ is based on the chemical fixation of carbonate minerals such as calcite, aragonite, and magnesite. The inventions described herein use a biomimetic CO₂ absorption mechanism using biocatalysts such as CA to reduce local CO₂ concentrations emitted from the production of cement.

Despite numerous attempts to pioneer functional concrete: use of enzyme-catalyzed calcium carbonate precipitation to repair concrete, the preparation of self-healing concrete containing bacteria, or the high cost of self-healing concrete containing capsule system, these cannot escape the scope of consuming concrete cement materials as the matrix. To fundamentally solve the CO₂-generating problem of concrete production, a viable substitute material for cement is required.

The technique of microbially-induced calcium carbonate precipitation (MICP) includes applying Synechococcus from photosynthetic cyanobacterium in MICP, to develop long-term viability building materials (LBMs), which present a successive regenerated ability and is an alternative to concrete cement material. However, the mechanical properties of LBMs are not comparable to natural concrete cement, and this material is far from practical construction applications. Additionally, the mechanism of crystal precipitation and growth within the bio-scaffold is not efficient thereby resulting in a material with low density. The bacteria may require maintenance to continue viability.

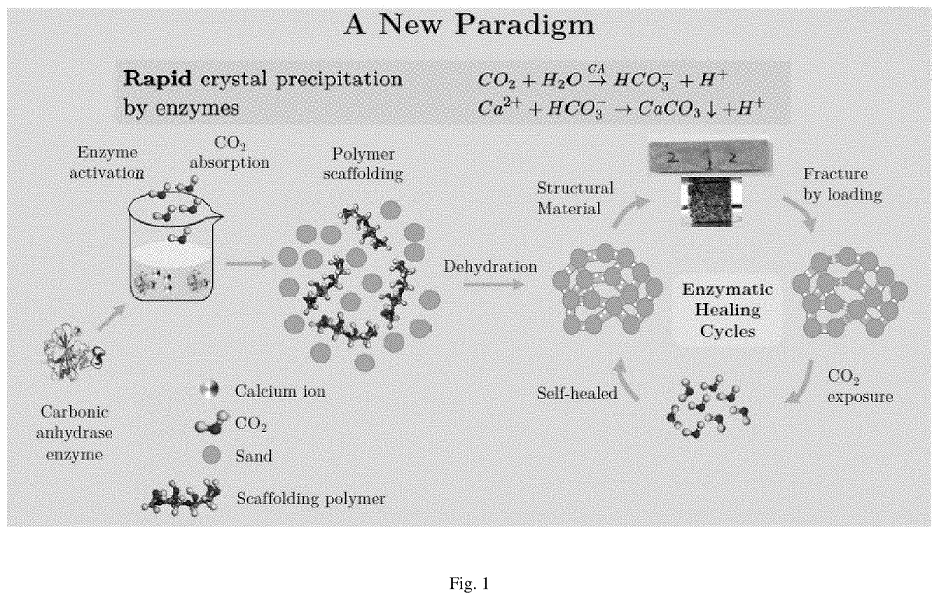

The examples described herein present a faster and more effective way to create a negative emission self-healing construction material using sand aggregates, a trace amount of enzyme, a small dosage of scaffolding material with a crosslinking agent, and a calcium source. Curing of the material can be performed at a high temperature for a short period or at room temperature for a longer period. This enzyme-driven method to bridge the sand particles results in a dense, stiff, strong, and relatively tough structural material, which upon exposure to calcium source and CO₂ can also heal itself repeatably. Carbonic anhydrase (CA), the enzyme used to generate the new material, can be easily stored and is stable at pH 6.5-8.5 and temperature of 30-50° C. The reaction conditions generate an environment that promotes enzyme stability, consumes CO₂, and avoids unhealthy reagents and pollutants. The mechanism of crystal growth on the scaffold yields outstanding mechanical properties and self-healing ability due to the catalyzing effect of CA. The enzyme-catalyzed process, structure fabrication process and 2D microstructure of the material, called enzyme construction material, ECM, are shown schematically in FIG. 1.

Concrete production and waste significantly contribute to global pollution. Enzymatic construction material (ECM) is a low-cost and environment-friendly construction material that possesses superior mechanical properties to concrete through enzyme-catalyzed crystal precipitation. The examples described herein show carbonic anhydrase (CA), an enzyme derived from biological cells, to facilitate the carbonation reaction by converting carbon dioxide to precipitate calcium carbonate crystals. In some embodiments, a synthetic analog of CA is used as the catalyst or enzyme. A polymer backbone (gelatin) provides a scaffolding framework for crystals to form and establish strong crystal bridges to connect the sand particles. The presence of enzyme molecules as catalysts allows ECM to possess self-healing capability. The examples described herein show that ECM endures six healing cycles, with damage at a specific location, with a loss of about 50% of overall strength. Therefore, ECM is a viable alternative to concrete, as the most used material in the world. ECM has limitless possibilities for green construction materials and the creation of space construction materials.

The examples described herein introduce a new paradigm in developing a negative-emission construction material with self-healing capability using sand, calcium, gelatin, and trace amount of carbonic anhydrase enzyme, named Enzymatic Construction Material or ECM. ECM possesses exceptional mechanical properties as a construction material with compressive strength (12 MPa) higher than any other available method and is twice as high as cement mortar. Therefore, ECM can be used to repair or even replace Portland cement concrete. The examples described herein examined the influence of CA enzyme catalysis, high pH condition, and gelatin on the overall strengthening and toughening mechanism in the ECM. The data show that wider and longer mineral bridges are incorporated in the microstructure of ECM compared to samples not containing enzyme and/or base samples. The bridges strengthen and toughen the material.

The self-healing capability of ECM was investigated through a cyclic fracture routine and healing examples, in which the ECM samples withstood up to six cycles of fracture. Therefore, the enzymatic mineralization method is useful to create a negative emission construction material and a method of carbon sequestration. The ECM can be rapidly manufactured with an environmentally friendly procedure, and therefore is a substitute for current building materials.

The ECM requires only a small amount of low-cost polymer as a scaffold and incorporates only trace amounts of an enzyme to produce an odorless, inexpensive, environmentally friendly, and mechanically strong material. The ECM can heal itself after multiple damage cycles at the same location demonstrating strong self-healing capacity. In some embodiments, the ECM is cured by baking at low temperatures. In alternative embodiments, the ECM is cured by light. In other embodiments, the ECM is cured by applying a magnetic field.

ECM is a negative emission structural material with the self-healing capability which further enhances its durability, and consequently, its negative carbon footprint. The material can be essentially used as an alternative carbon sequestration method. ECM alleviates the high monetary and energy costs associated with the production and use of concrete and consumes the greenhouse gas carbon dioxide. Most important, ECM does not use any harmful reagents with foul odors that would limit the application of the final structure. Therefore, ECM provides a novel, low-cost, safe, and highly efficient way to create a sand slurry material with a strong self-healing capability. ECM is a new material and method for the development of environmentally friendly construction materials. Therefore, Enzymatic Self-Healing Construction Material is negative emission material that consumes CO₂ during production, and during its self-healing process.

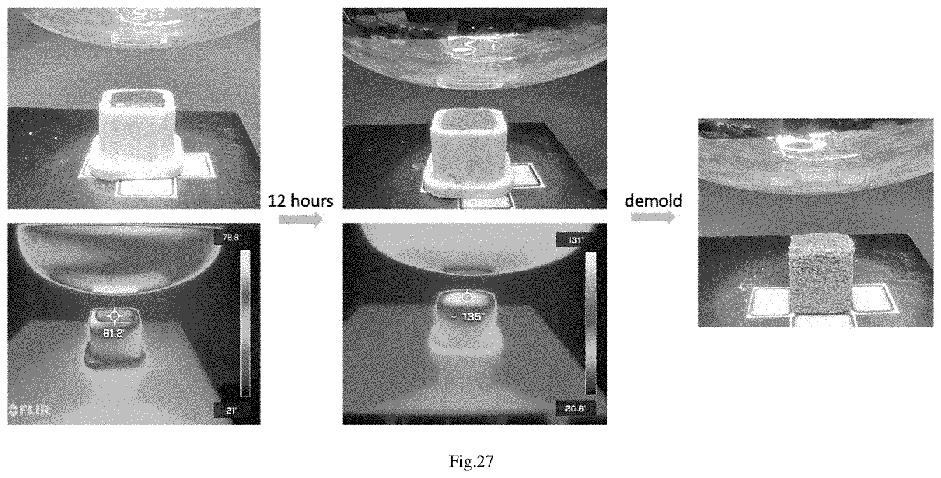

Traditional concrete curing requires an adequate amount of moisture for continued hydration and 28 days to achieve mechanical strength. Compared to concrete, ECM obtains maximum compressive strength in 24 hours by oven heating or more than one week under natural desiccation. However, to reduce the time cost, the application of the oven makes the on-site construction challenging. Embodiments of the invention described herein show that adding a trace amount of iron oxide nanoparticles to ECM generates an improved material called ECM-n that is cured with a low-power laser, or incandescent light (FIG. 27). This method, based on studies of laser-induced nanoparticle application in hyperthermia therapy utilizes the exothermic behavior of iron oxide nanoparticles under external electromagnetic (radiofrequency, microwave, and laser) excitation. Specifically, the ECM-n samples cured for 12 hours under a 3W laser (808 nm wavelength) have similar compressive strength to ECM. Additionally, fabricating large flaws in ECM-n samples showed accelerated repair with laser exposure and the samples regained mechanical strength and properties comparable to original, flawless samples.

A portion of the embodiments described herein was published in Matter Volume 5, Issue 3, Mar. 2, 2022, Pages 957-974 as “A self-healing enzymatic construction material” by co-inventors Shuai Wang, Suzanne F. Scarlata, and Nima Rahbar, which is hereby incorporated by reference herein in its entirety. Another portion of the embodiments described herein are submitted as a manuscript to Cell Reports Physical Science as “Curing and self-healing of enzymatic construction materials using nanoparticles” by o-inventors Shuai Wang, Suzanne F. Scarlata, and Nima Rahbar, which is hereby incorporated by reference herein in its entirety.

The inventions described herein are the most practical methods. It is recognized, however, that departures may be made within the scope of the invention and that modifications will occur to a person skilled in the art. Concerning the above description then, it is to be realized that the optimum dimensional relationships for the parts of the invention, including variations in size, materials, shape, form, function, steps, and manner of operation, assembly, and use, would be apparent to one skilled in the art, and all equivalent relationships to those illustrated in the drawings and described in the specification are intended to be encompassed by the present inventions.

The following examples and claims are illustrative only and not intended to be further limiting. Those skilled in the art will recognize or be able to ascertain using no more than routine experimentation, numerous equivalents to the specific procedures described herein. Such equivalents are within the scope of the present invention and claims. The contents of all references including issued patents and published patent applications cited in this application are hereby incorporated by reference.

The invention now having been fully described, is further exemplified by the following examples and claims.

### Example 1: Enzymatic Solution Preparation

The analytical grade carbonic anhydrase (CA), calcium chloride dihydrate, and tris (hydroxymethyl) aminomethane (THAM) were obtained from Sigma Aldrich chemical company. To prepare the enzyme matrix solution, 2.9 mg CA and 1 mL deionized water were mixed into aliquots, then shaken evenly in the horizontal direction for two minutes. The CA solution (5 L of 100 M CA) was pipetted in each of the empty aliquots and stored in the freezer.

### Example 2: Calcium Carbonate Solution Preparation

To prepare a buffer solution, 0.1M tris (ordered from Sigma Aldrich) was added to 200 mL deionized water, mixed with 2M saturated solution, and stirred for two minutes. Calcium chloride dihydrate was chosen as the calcium source because it can generate calcium carbonate more efficiently. A high concentration of free calcium ions in the solution expedites the dissolution of calcium chloride dihydrate. When the calcium chloride dihydrate was dissolved substantially, 10 L of 100M CA solution was added into a 1000 mL beaker and stirred for another two minutes. CO₂ gas was then introduced into the solution at a rate of five bubbles per second for 10 minutes. A pH meter was used to record the entire process. Some fog was observed to be produced from the solution after five minutes. The experiment ended when the pH reached seven and the flow of CO₂ gas was stopped. The solution was then settled for four hours, and the supernatant was siphoned to prepare a highly concentrated calcium carbonate solution.

### Example 3: Cubic and Cuboid ECM Sample Fabrication

Cubic samples 25 mm were prepared for uniaxial compressive tests. A loading rate of 1.27 mm/s was chosen. Single-edge notch beam samples were prepared for the fracture test and plain beam for the bending test. 10% gelatin by weight (gelatin/solution) was mixed with sand for two minutes. Gelatin was chosen because of its chemical compatibility with CA, and because the completely soluble temperature of gelatin is 35° C., which is within the temperature range of carbonic anhydrase activity. The sand-gelatin mixture was placed in silicone molds. The calcium carbonate solution was then titrated on the sample surface. The samples were allowed to settle for half-hour until the solution was completely saturated in the matrix. The ECM samples were then desiccated in the 100° C. oven for 24 hours and demolded for testing.

### Example 4: Weight Gain

Four groups of solutions were prepared (Ca: Ca²⁺; BCa: Base+Ca²⁺; BEC: Base+CA+Ca²⁺; ECa: CA+Ca²⁺) similar to the method described in Enzymatic solution preparation. The solution was then aerated with CO₂ gas (5 bubbles per second) for 10 minutes with continuous stirring. The weight of the solution was measured immediately after the end of the experiment.

### Example 5: Self-Healing Experiment Procedure

To examine the self-healing capability of ECM, single-edge notch beam samples were prepared similar to the method presented in the Cubic and cuboid samples fabrication process section. The first cycle was initiated with the beam samples that were fractured under three-point bend test and then placed back into the silicon mold. To demonstrate the self-healing process, a constant amount of 2 mL of calcium-enzyme solution was titrated evenly on the surface of the crack region. Then CO₂ gas was aerated through a rubber tube with constant pressure on the crack surface for 10 minutes. Samples were then desiccated in the 100° C. oven for 24 hours and then underwent the three-point bending fracture test. The fracture/healing procedures were repeated six times and the data were collected.

### Example 6: Glutaraldehyde Modification

Glutaraldehyde 50% concentration was acquired from TCI AMERICA company as the ECM crosslinking agent. Gelatin and glutaraldehyde at a 1:1 ratio were then added by weight on the surface of the ECM samples. The samples were placed at room temperature for half an hour until the gelatin/glutaraldehyde solution completely penetrated the sample. The samples were then desiccated for another 24 hours and then mechanically tested.

### Example 7: X-Ray Diffraction (XRD) and FT-IR Analysis

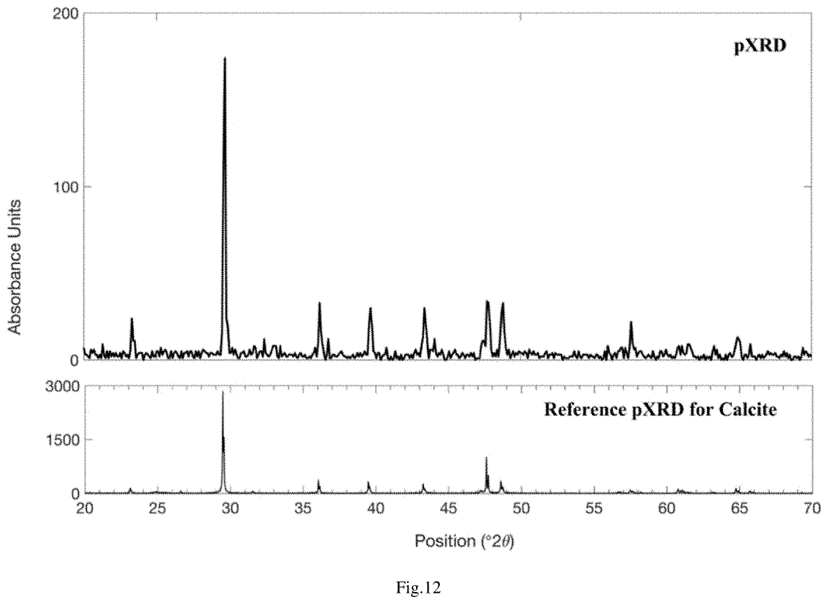

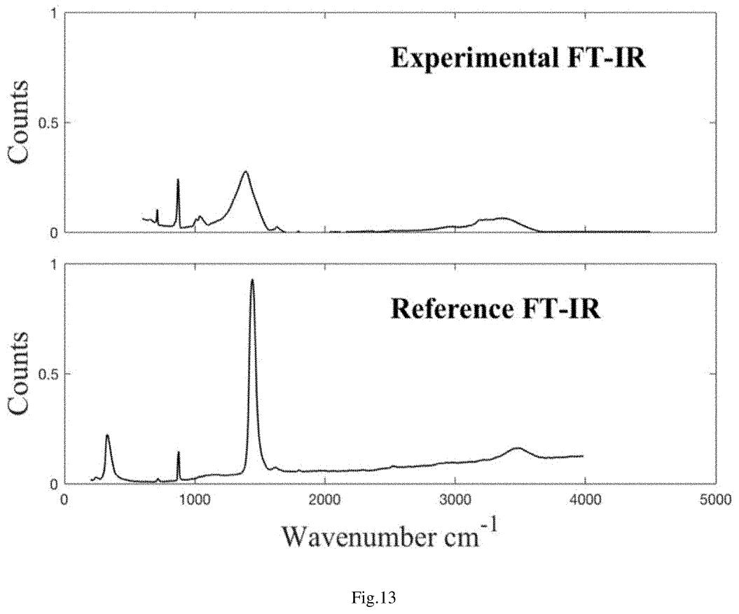

Analysis of the dried enzyme solution product was conducted by powder X-ray diffraction (pXRD) and Fourier Transform Infrared Spectroscopy (FT-IR) to confirm both the chemical composition and crystallinity. pXRD was performed on a Bruker AXS D8 Focus (Bruker, Billerica, Mass., 102 of 190 USA) at 25° C., and the pXRD spectrum used a CuKa radiation source at 40 keV and 40 mA from 20° to 90° of 2θ with a step size of 0.100°, against the baseline and FT-IR was carried out on a Bruker Optics Vertex 70 equipped with a Specac Golden Gate Diamond Single Reflection ATR element (Bruker, Billerica, Mass., USA). The calcium carbonate prepared from the enzyme solution was rinsed and dried for analysis. FIG. 12 compares the pXRD spectrum against the reference spectrum for calcite, and FIG. 13 compares the FT-IR spectrum of the dried enzyme product against the baseline FT-IR spectrum for Ca-carbonate obtained from the RUFF Project database. Both results verify the chemical composition and crystallinity of the enzyme product as calcite.

### Example 8: Compressive Strength of Cubic Coarse Sand ECM

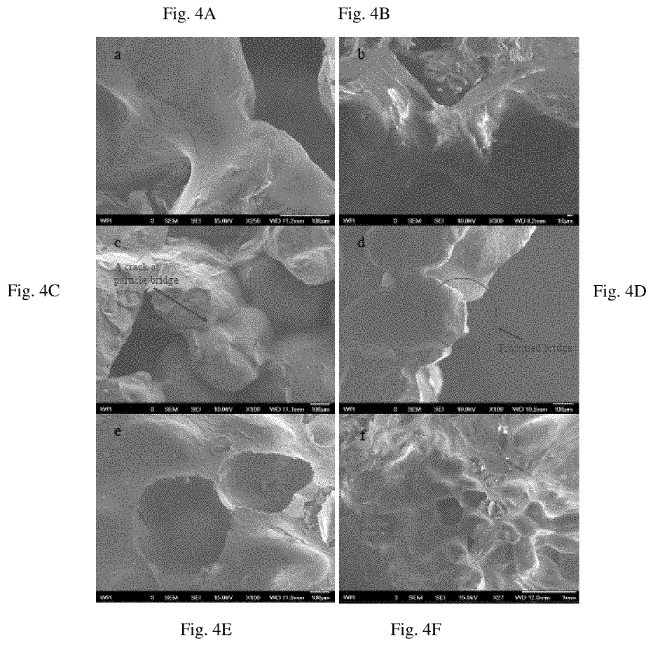

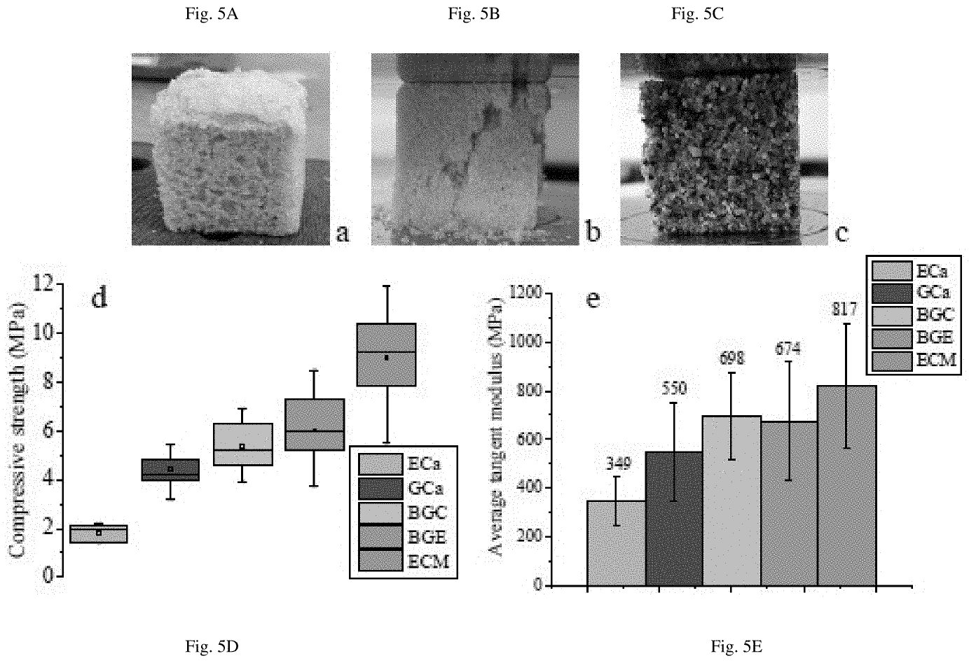

The uniaxial compressive experiments were performed following the American Society for Testing and Materials (ASTM) standards on ECM specimen with coarse sand (NO. 30, the sand retained on a 600 μm sieve as alternative sand mixing in the materials), since ECM design possessed the highest compressive strength compared to intact and high pH specimens in the extensive fine sand experimental plan. The average strength of coarse sand ECM is 4.2 MPa which is lower than fine sand ECM (FIG. 4D). The primary reason is that compressive strength in these samples is predominantly influenced by larger sand aggregate strength, size, and distribution than the size of crystal bridges.

### Example 9: Fracture and Bending Test

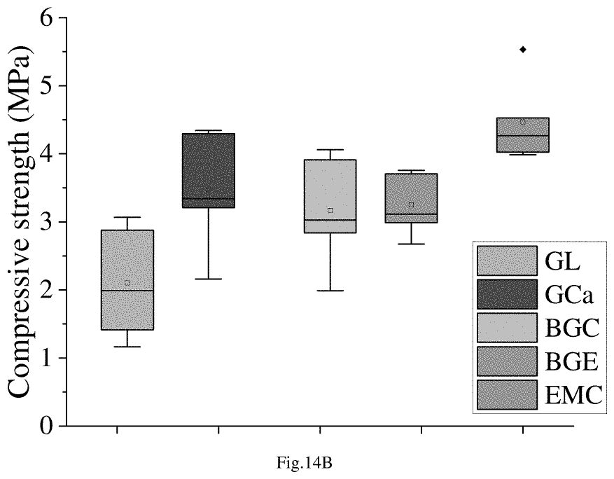

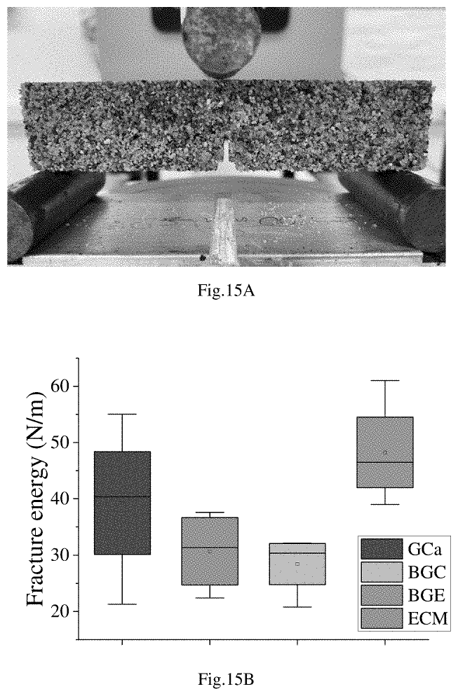

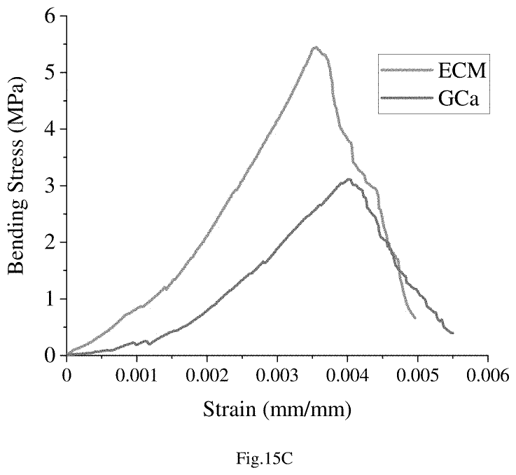

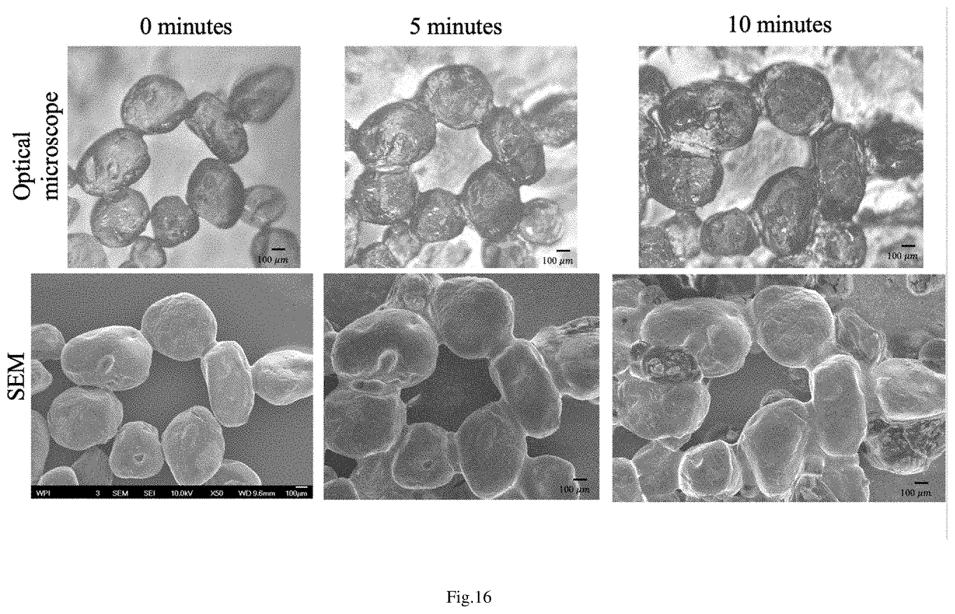

The results of the three-point fracture experiments are presented in FIG. 15A-FIG. 15C. The fracture experiments were followed using the method in ASTM standard. A cured silicone mold with a sharp crack was prepared and used for sample preparation. A four-part solution of 10 ml CA enzyme, tris base, 2M Calcium chloride dihydrate and DI water was prepared. It was then thoroughly mixed for two minutes and bubbling CO₂ gas for 10 minutes. The solution was then titrated on the coarse sand samples and settled for half hour until fully infiltrated into the sand. The samples were then placed in the 100° C. oven for 24 hours and demolded afterward. Fracture toughness was then calculated following the fracture experiments. Higher fracture energy indicated materials undergo more ductile failure. The data show that the coarse sand ECM sample has the highest average fracture energy (218 N/m).

### Example 10: Crystal Bridge Growth Mode

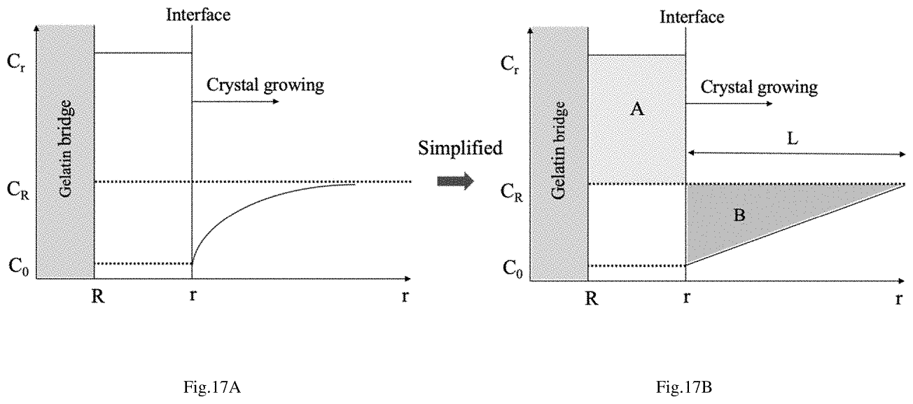

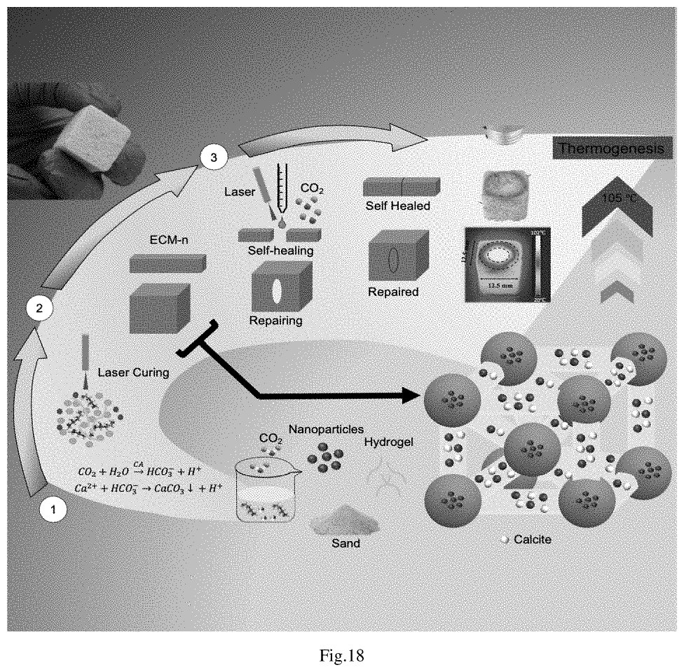

In an oversaturated calcium chloride solution, the gelatin scaffold provides a stable platform for crystal nucleation and growth. The solute calcium carbonate then continues to precipitate around bridges which causes the surrounding concentration of calcium carbonate lower than the entire system. The solute CaCO₃ precipitate at the gelatin surface is denoted as C_{R}. The molar concentration of crystals in the solid mineral is represented by Cᵣ. The molar concentration of CaCO₃ in the solution right around the solid mineral is C₀, which is very low. The dissolution of calcium carbonate is difficult since its solubility is only 13 milligrams per liter. The concentration profile is shown in FIG. 17A. Three defined concentrations follow a relationship written as Cᵣ>C_{R}»C₀. Now, the number of molecules CaCO₃ (dN) that are moving toward the interface to form the mineral bridges are calculated using Fick's first law within a time interval, dt, as

$$d N = D ( 2 π r d z ) \frac{\partial C}{\partial r} d t \qquad ( 1 )$$

where, D is the diffusion coefficient, dz is the length of the element along the bridge's direction, is the distance from the center of the lattice beam and C(r,t) is the instantaneous concentration of the solution.

For the mineral bridge interface to advance a distance dr within a time interval dt, the change in the number of solute CaCO3 molecules within the mineral bridge is written as

dN=(Cᵣ−C₀)(2πrdz)dr (2)

By equating Equations 2 and 3, the following equation is obtained

$$\frac{dr}{dt} = \frac{D}{{C}_{r} - {C}_{0}} \frac{\partial C}{\partial r} \qquad ( 3 )$$

The ∂C/∂r is further estimated by a simplified linear concentration profile as shown in FIG. 17B as:

$$\frac{\partial C}{\partial r} = \frac{{C}_{R} - {C}_{0}}{L} \qquad ( 4 )$$

Therefore, by equating the molar numbers of CaCO₃ in these two shaded areas A and B,

$$π ( {r}^{2} - {R}^{2} ) d z ( {C}_{r} - {C}_{R} ) = π [ {( r + L )}^{2} - {r}^{2} ] d z ( \frac{{C}_{R} - {C}_{0}}{2} ) \qquad ( 5 )$$

L is obtained as follows:

$$L = \sqrt{{r}^{2} \frac{2 {C}_{r} - {C}_{R} - {C}_{0}}{{C}_{R} - {C}_{0}} - {R}^{2} \frac{2 {C}_{r} - {C}_{R}}{{C}_{R} - {C}_{0}}} - r \qquad ( 6 )$$

Using the above equations, the growth rate of a mineral bridge is derived as

$$\frac{dr}{dt} = \frac{D ( {C}_{R} - {C}_{0} )}{( {C}_{r} - {C}_{0} ) ( \sqrt{{r}^{2} \frac{2 {C}_{r} - {C}_{R} - {C}_{0}}{{C}_{R} - {C}_{0}} - {R}^{2} \frac{2 ( {C}_{r} - {C}_{R} )}{{C}_{R} - {C}_{0}}} - r )} \qquad ( 7 )$$

and since C₀ is negligible, it is further simplified to

$$\frac{dr}{dt} = \frac{D {C}_{R}}{{c}_{r} ( \sqrt{{r}^{2} \frac{2 {C}_{r} - {C}_{R}}{{c}_{R}} - {R}^{2} \frac{2 ( {C}_{r} - {C}_{R} )}{{c}_{R}}} - r )} \qquad ( 8 )$$

If the mineral bridge thickness is defined as H=r−R, Equation 9 can be rewritten as:

$$\frac{d ( H / R )}{d ( Dt / {R}^{2} )} = \frac{1}{\frac{{C}_{r}}{{C}_{R}} \sqrt{{( H / R + 1 )}^{2} ( \frac{2 {C}_{r}}{{C}_{R}} - 1 ) - ( \frac{2 {C}_{r}}{{C}_{R}} - 2 )} - \frac{{C}_{r}}{{C}_{R}} ( H / R - 1 )} \qquad ( 9 )$$

The initial condition is H(t=0)=0. Now, if we set Cᵣ/C_{R}=α, H/R=Y, and Dt/R²=X, therefore:

[α\sqrt{(Y+1)²(2α−1)−(2α−2)}−αY−α]dY=dX (10)

Finally, by setting the boundary condition, when t=0, the X=0, and integrating Equation 10 from 0 to X:

$$\frac{a ( a - 1 )}{\sqrt{2 a - 1}} \{ [ ( Y + 1 ) \sqrt{\frac{2 a - 1}{2 a - 2}} \sqrt{\frac{2 a - 1}{2 a - 2} {( Y + 1 )}^{2} - 1} - ln ( \sqrt{\frac{2 a - 1}{2 a - 2} {( Y + 1 )}^{2} - 1} + ( Y + 1 ) \sqrt{\frac{2 a - 1}{2 a - 2}} ) ] - [ \frac{\sqrt{2 a - 1}}{2 a - 2} - ln ( \sqrt{\frac{1}{2 a - 2}} + \sqrt{\frac{2 a - 1}{2 a - 2}} ) ] \} - α ( \frac{{Y}^{2}}{2} + Y ) = X \qquad ( 12 )$$

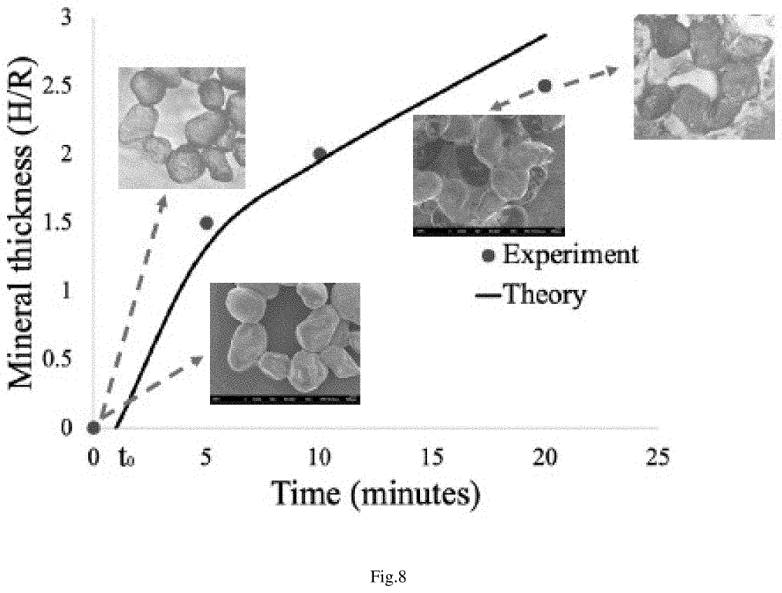

Therefore, crystals start to precipitate in a few seconds, thus the initial time to cannot be equal to zero. The experimental parameters assumed: R=200 μm, α=4.8 and D=1.6×10⁻⁹ m²/s. The mineral bridge thickness as a function of time is plotted in FIG. 8. The experiment results agree with the presented model.

### Example 11: Reinforced ECM-Tannin Modification

The 0.8% by sample weight of tannin was added to the ECM. This value is significantly higher than the regular ratio in gelatin system modification to achieve a stronger crosslinking. The gelatin-tannin systems can form more and stronger bonds at a faster rate than gelatin systems. However, the performance of crosslinking bonds in compression is not as good as in tension. The powdered wine tannin from Midwest Homebrewing and Winemaking Supplies. Inc was selected Tannin 28% of gelatin weight was manually mixed with gelatin and sand particles and stirred for two minutes. Calcium carbonate solution was prepared in similar fashion described for prior experiments and heated up to 35C. The matrix was then poured into the cubic silicon mold and 7 mL solution was pipetted on each cube. The samples were then desiccated before conducting the compression test.

### Example 12: Catalytic Performance

CA is the fastest known enzyme with a high rate of catalysis of CO₂ in a diffusion-controlled process. The method of creating ECM is based on CA catalyzing the rapid precipitation of CaCO₃ to create mineral bridges that connect the sand aggregates in two separate steps: CA first catalyzes the reversible hydration of carbon dioxide to bicarbonate (Equation 13) which then reacts with calcium ions in the solution to precipitate CaCO₃ (Equation 14). Synthetic CA analogs mimic the CA reaction. However, the synthetic analogs may be less efficient and would have to be used at higher concentrations to achieve similar results.

The efficiency of the reactions depends on four key factors: (1) the concentration of calcium available for the nucleation site. The catalysis by the CA enzyme shows a high biomineralization efficiency where the calcite crystals (CaCO₃) self-assemble into a sand-gelatin matrix to establish ‘bridges’ between the sand particles. Gelatin serves here as a harmless and odorless scaffold, which gains strength by physical crosslinking after dehydration. It can be regarded as a carrier of crystal, connecting the whole microstructure system.

$${CO}_{2} + {H}_{2} O {⟶}^{CA} {HCO}_{3}^{-} + {H}^{+} \qquad ( 13 )$$

$${Ca}^{2 +} + {HCO}_{3}^{-} ⟶ {CaCO}_{3} {↓}^{+} {H}^{+} \qquad ( 14 )$$

There is enough ambient carbon dioxide in the air to drive the main chemical reaction prescribed earlier. While the reaction between carbon dioxide and water is reversible, both sides of the reaction proceed simultaneously to achieve a “dynamic equilibrium”, which hinders the precipitation of the calcium carbonate crystals. In this process, carbon dioxide dissolves in water to form carbonic acid, while chloride ions, from calcium chloride, produce hydrochloric acid, and as the reaction proceeds, calcium carbonate and hydrochloric acid are formed. Buffer is often added to prevent a drop in pH and enhance calcium carbonate crystal precipitation. According to Le Chatelier's principle, the balance moves in the direction of reducing hydrogen ions. In other words, the direction is moving toward producing weak acid molecules. Conversely, when a weak acid is added to a strong acid salt solution, since the strong acid has no ionization equilibrium, the change in the concentration of hydrogen ions in the solution will not create strong acid. Therefore, when the solution is proportioned, to make the calcium carbonate crystal precipitate constantly, a tris buffer is added to the reaction. The buffer solution is composed of the weak acid “HA”, and its own salt “NaA”.

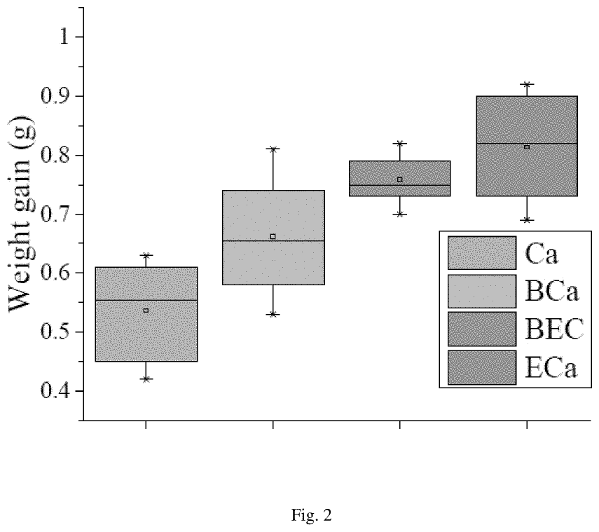

The buffer solution contains enough alkali “A⁻” ions used to buffer the strong acid in the solution. When a certain amount of strong acid is added to the solution, H⁺ ions are consumed by “A⁻” ions, and the pH value of the solution is constant. Thus, the calcium carbonate crystals can precipitate successfully. As a first step in developing the material, the efficiency of CA was quantified by measuring the weight gain from the precipitated crystals produced in the enzymatic reaction. The results of weight gain from four different solutions are presented in FIG. 2. ‘Ca’ represents the aqueous solution that includes only the calcium source; ‘BCa’ represents the base and calcium source solution; ‘BEC’ represents the base, CA enzyme and calcium source solution; ‘ECa’ represents the system of the sand slurry without gelatin, in which only CA enzyme and calcium source were added into the solution.

These data show that within the same time range, the ECa sample obtained the highest average weight gain of about 0.82 g, which is higher than the BEC samples about 0.07 g, and significantly higher than the Ca samples about 0.24 g. The results indicate that the carbonation by enzyme catalyzing is efficient and the calcium crystallization ability in ECa is the highest in the group. It should be noted that the weight gain is not equal to the amount of precipitated crystal precipitation but indirectly reflects the catalytic ability of the enzyme to convert carbon dioxide. The weight gain in every solution is equal to the quantity of absorbed CO₂, which is used to calculate the weight of precipitated calcium carbonate based on Equations 13 and 14. The catalytic efficiency K_{cat} of CA at 25° C. and pH=9 is about 106 rec/s, which, defined as the CO₂ hydration turnover rate, is the maximum number of substrate molecules transformed into the product by a single CA in one second.

TABLE 1
A comparison of the CA enzyme catalyze
activity in experiment and theory

| K_{cat} = 10⁵ rec/s | Weight gain (g) | CaCO₃ (g) | CO₂ (Molecules) |
|---|---|---|---|
| Experiment | 0.82 | 1.86 | 1.12*10²² |
| Theory | 2.56 | 5.80 | 3.49* 10²³ |

The theoretical and experimental results are compared in Table 1. The theoretic result for CO₂ molecules is 3.49*10²³ and the experimental molecules quantity of CO₂ molecules is 1.12*10²², considering the experimental error and the effect of carbon dioxide on pH (K_{cat} will change by pH), the experimental results are well in agreement with the predicted results in the ambient temperature regime, and the enzyme indeed performed an improvement in carbonation.

Comparing the BCa samples in FIG. 2 with Ca samples, it was observed that more crystals precipitated after adding a base reagent to the solution. From Equation 13, the hydroxide ions neutralize the hydrogen ions in the solution and push the reaction forward. The urease uses the nitrogen source, urea, to produce hydroxide ions to increase the solution pH, causing calcium carbonate to precipitate from the calcium ions in the solution.

The CA-mediated calcium carbonate precipitation was compared to the urease method, and the comparisons included mechanical properties and structure formation. The calcium carbonate without sand formed by the urease slurry is a non-uniform crystal structure, however the calcium carbonate from the CA slurry is organized and uniform in layers. The calcium carbonate in the CA method adheres to the sand particles and forms an organized, uniform layered structure, most likely due to the finding that faster production of the precipitates decreases the mobility of solid particles. Additionally, in terms of mechanical properties, the CA samples outperform urease samples in both parameters of the compressive strength and tangent modulus. These experiments indicate that the CA materials and methods of use are more effective in mineral precipitation and the assembly of sand slurry construction materials than other materials tested.

### Example 13: Microstructural Characterization

To understand if the composition and morphology of ECM are influenced by the presence of the CA enzyme, four different compositions were used to assess the quantity of mineral precipitation, mineral distribution, structural arrangement, and bridge size different at high pH, no CA enzyme and no calcium source. The control ‘GL’ (Gelatin) samples were created with the same media as the ECM (CA+Gelatin+Ca²⁺), but only containing gelatin. The ‘BGE’ (Base+gelatin+CA+Ca²⁺) represents a high pH control group, where higher pH helps to produce more partials of calcium carbonate. The ‘GCa’ (Gelatin+Ca²⁺) samples demonstrate the effectiveness of the CA enzyme in crystal precipitation and microstructure of the resulting product which was fabricated with the gelatin and calcium source.

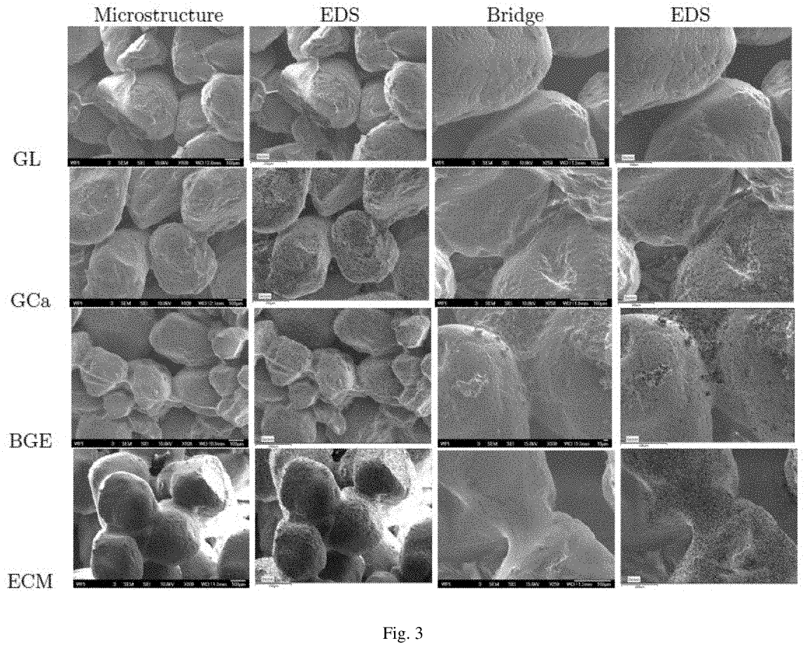

Compared to the other three groups, it was observed that the structure of ECM is denser and more organized. The surfaces of GL and GCa samples were found to be smooth, and their bridges in some domains were too short or too fine. The mineral bridges in BGE are stronger, but the overall arrangement of structure is inordinate. During the preparation of ECM, most of the calcium carbonate crystals were trapped or covered in the gelatin and formed a bridge, and it is difficult to observe the crystal distribution of the surface directly through SEM. The samples were then analyzed by Energy Dispersive X-Ray Spectroscopy (EDS). EDS can qualitatively determine and locate the crystals by the content of calcium. The results of the distribution of Ca2+ from EDS analysis are shown in FIG. 3. There is no calcium observed in the EDX maps of the GL samples since no calcium was added. EDS revealed a dense distribution of calcium in ECM that contributes to the broader and wider bridges which cover a large portion of the surrounding sand particle surfaces. In samples GCa and BGE, only a small amount of calcium is distributed on either sand surfaces or bridges. FIG. 4A and FIG. 4B display a close view of the bridging mechanism between ECa (CA+Ca2+) and ECM samples. Although the calcium carbonate crystals form a decent bridge network to bridge the sand particles, the size of the ECa bridge (around 80 m) is smaller than the ECM (150 m), which means that the gelatin scaffolding enhances and extends the bridges. The crystals align closely in gelatin resulting in an organized and dense structure that establishes a mechanism for the enhancement of the mechanical properties of the ECM and provide the physical basis for increased durability and corrosion resistance of the resulting product.

The fracture mechanism of ECM relies on the bridging connections among the sand aggregate components. FIG. 4C and FIG. 4D show the ECM sample after mechanical testing, displaying the cracks that propagate in the center of the bridges in the transverse direction. FIG. 4D shows a typical fractured surface at the crack tip region where a 150 m diameter fracture surface is exposed. FIG. 4E shows sand aggregates were detached from the matrix; this is the other major fracture mechanism after bridge failure and was found within the sand gelatin system. However, this mechanism occurs more often in the case of self-healing cycles.

### Example 14: X-Ray Diffraction (XRD) and FT-IR Analysis

To examine the crystallinity and chemical composition of the ECM enzyme product, powder X-ray diffraction (pXRD) and FT-IR were performed on a Bruker AXS D8 Focus (Bruker, Billerica, Mass., 102 of 190 USA) at 25° C., the pXRD spectrum using a CuKa radiation source at 40 keV and 40 mA from 20 to 90 of 2 with a step size of 0.100, against the baseline and Bruker Optics Vertex 70 equipped with a Specac Golden Gate Diamond Single Reflection ATR element (Bruker, Billerica, Mass., USA). The results (FIG. 12 and FIG. 13) verify that the crystallinity of the enzyme product consists mostly of calcite.

The mechanical experiments were focused on the compressive strength, tensile and fracture properties of ECM with reference groups. Compressive tests and three-point bending tests were performed to study the mechanical performance of the proposed enzymatic construction material. Five different groups of sand slurry samples were prepared and termed: ECa (CA+Ca²⁺), GCa (gelatin+Ca²⁺), BGC (Base+Gelatin+Ca²⁺), BGE (Base+Gelatin+CA+Ca²⁺), and ECM (CA+Gelatin+Ca²⁺). It is noted that the above samples incorporate the calcium source. The compressive strength and elastic moduli are presented in FIG. 5A-FIG. 5E.

The main function of construction materials such as concrete is compressive strength. The compressive strength of the concrete mixture depends on the properties of the aggregate and cement mortar matrix. As a potential construction material, ECM also possesses its own ‘aggregate’ and ‘mortar matrix’, and its mechanical properties are similarly a function of the strength of sand and mineral bridge. The results in FIG. 5A show that gelatin as scaffold in GCa can increase the compressive strength of the sand slurry material ECa. However, in comparing GCa with ECM, an average strength of 4.5 MPa is not noticeable. The data show that the ECM performs with the highest compressive strength and elastic modulus.

The average compressive strength of the ECM samples is 9 MPa, which is twice as high as the GCa specimens, 4.7 times greater than the ECa specimens, and significantly higher than the base amended specimens of BGC & BGE (FIG. 5D). The elastic modulus of ECM is higher than other specimens, which means the elastic deformation under the same external force is smaller. It can be observed from EDS-SEM images in FIG. 3 that the size of crystal bridges in the ECM on average is about 150 m wide and 50 m long. Also, the calcium carbonate crystals in ECM are distributed more uniformly. Comparing the EDX maps of ECM samples with BGE and BGC samples, a significant amount of calcium was observed on the ECM bridges. Therefore, compared with the structure and mechanical properties of the other three groups, it can be deduced that the crystal bridges are the principal strengthening and toughening mechanisms in these heterogeneous materials.

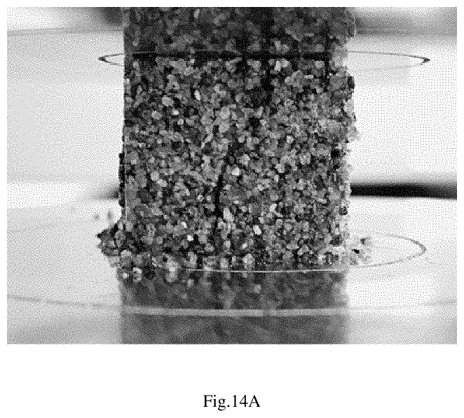

It is noted that the compressive strength of ECM is more than two times of standard cement mortar (with a specific strength of 3.5 MPa). The sand slurry cubic samples with coarse aggregates were also fabricated (FIG. 5C), and their compressive strength is presented in FIG. 14. The coarse sand slurry ECM performs a higher average strength than high pH BGE samples by about 31%, and control gelatin samples GCa by about 17%. CA enzymes play the same role in both fine and coarse sand samples by increasing the rate of crystal precipitation within a fixed time, and consequently, strengthening the samples. However, the maximum size of the crystal bridges is limited by the amount of calcium, which prevents the increase in strengthening in coarse aggregates above a certain threshold. The maximum size of bridges limits the challenge of bonding large sand particles, which results in relatively lower strength for coarse aggregate samples than for fine aggregate samples. Since the size of the crystal bridges in the fine aggregate samples is comparable to the size of the particles, the forces are more evenly distributed in the microstructure of the materials, and hence, higher overall strength and fracture energy, contributing to a product with greater durability.

### Example 15: Tensile Test

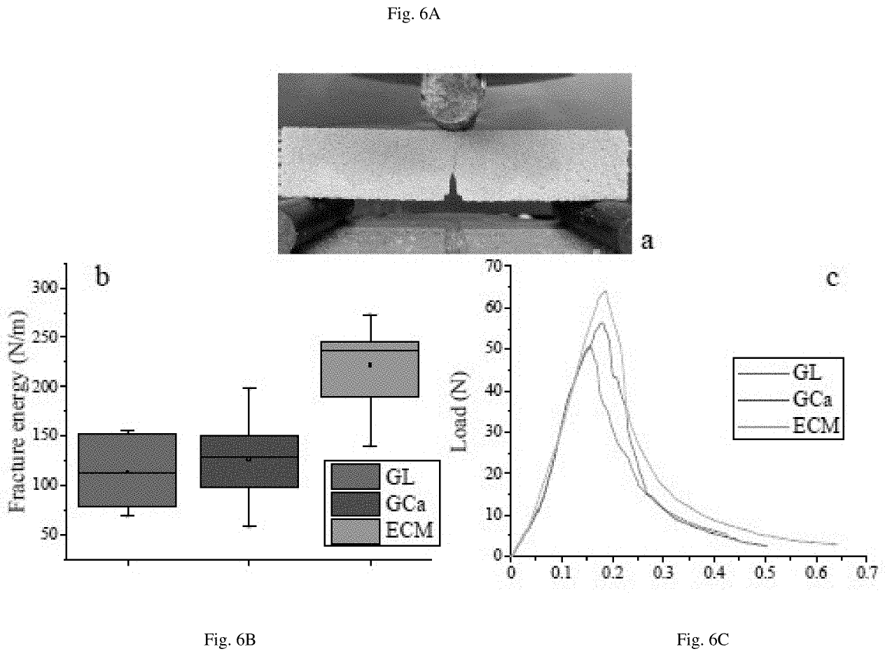

Fracture energy is an important mechanical property of building materials since the propagation and control of cracks are highly related to the serviceability and durability of materials. The single-edge notch bending (SENB) test of ECM beam samples, shown in FIG. 6A, was conducted and the results of the fracture tests are presented in FIG. 6B and FIG. 6C. Fracture energy and load-displacement curves reflect the excellent mechanical properties of ECM. FIG. 3 EDS maps show that the bridges are mainly calcium carbonate crystals incorporated within the gelatin scaffold to bridge the sand particles. Fracture surfaces in FIG. 4A-FIG. 4E also show the typical fracture mechanism in ECM by particle debonding and bridging failure. Hence, the toughening mechanism is mainly undertaken by mineral bridges. Therefore, an ECM with wider and longer bridges can exhibit excellent tensile properties. The fracture energy results indicated that applying the CA enzyme method significantly increases the fracture energy by about 100% compared with GL and by about 77% compared with GCa. The average ultimate load of ECM samples is also higher than GL samples by about 27% and GCa samples by about 16%. The fracture and bending tests of coarse sand slurry ECM are illustrated in FIG. 15A-FIG. 15C. The fracture energy of coarse sand slurry ECM is higher than control ‘GCa’ by about 38%, and higher than high pH samples (BGC) by about 120%. There is no other method that creates a structural sand slurry material with similar compressive strength and fracture energy.

In sand slurry materials, calcium carbonate crystals bridges are the critical element in providing compressive strength and play an interlocking role in the toughening mechanism. However, the polymer scaffold provides the main toughening system. These elements provide the improved durability of the ECM compared to earlier materials. Comparing the ECM beam fracture specimens with two control “GL” and “GCa” groups, it was observed that the fracture energy and ultimate tensile loads are promoted by CA enzymatic catalysis, and aggregate type and size can also affect the strength and fracture energy of these materials. However, similar trends were observed in the examples on coarser aggregate ECM. In coarse sand ECM samples, the mineralized gelatin matrix has a significant toughening role. The control groups without CA treatment (GCa) show lower fracture energy. Under high pH conditions, CA enzyme catalysis has a more negligible effect on the fracture energy, especially in coarse sand slurry samples (FIG. 15A-FIG. 15C). Although more crystals were precipitated, the impact of crystal quantity on the toughness of the coarse sand ECM may be less than other factors. In FIG. 15A-FIG. 15C, it is observed that more crystal precipitation does not increase the toughness of the material in the case of high pH. SEM images show that the calcium-gelatin matrix bridges of ECM are wider and longer than that of non-CA enzyme samples GCa, which plays an essential role in the toughening mechanism in these materials where fracture initiates on the bridges in the high-stress regions. However, since the mortar on the macro-scale is uneven, there may be other mechanisms to strengthen and toughen the specimens. The interfacial fracture toughness between the crystal bridges and the sand surface, which are a function of the surface roughness of the sand particles, can also affect the overall strength and toughness of these materials.

Although, previous publications state that a high pH condition can benefit the extent of carbonation, the overall performances of samples with the high pH preparation in the tests herein are not as good as the performances of the ECM samples. Within a limited reaction time, adding base reagents generates a portion of the calcium hydroxide with calcium carbonate in the precipitation. Calcium hydroxide consists of one calcium cation and two hydroxide anions at the molecular scale, and calcium carbonate consists of one calcium cation and one carbonate anion, in which both calcium hydroxide and calcium carbonate are non-polar. Most of the calcium carbonate (calcite) molecules possess a cubic structure at room temperature, which creates a more stable and stiff structure in all directions. However, calcium hydroxide crystals have a plane structure at room temperature and are weaker. The elastic modulus and strength of calcium carbonate are about 70 GPa, which is higher than that of calcium hydroxide at about 48 GPa, respectively. Therefore, the calcium hydroxide crystal in the bridge results in an inferior advantage compared to calcium carbonate for both compression and tension.

### Example 16: Crystal Growth Modeling

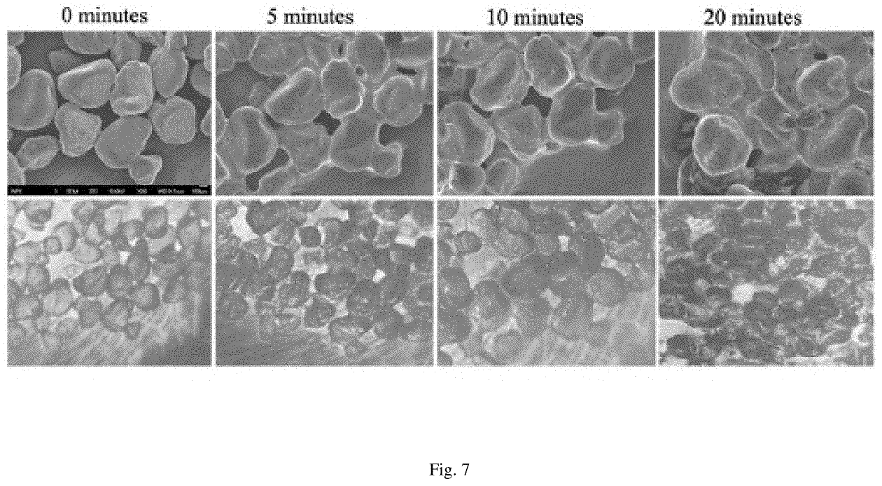

Understating the crystal nucleation and growth are of prime importance in determining and predicting the physical properties of ECM. Hence, optical and electron microscopy were extensively utilized to study the time-lapse of crystal growth in ECM samples. In FIG. 7, the formation of calcium carbonate crystals on the sand-gelatin system surfaces is analyzed using SEM and optical images. In the ECM, the rough surface of the gelatin scaffold and sand provides numerous sites for the nucleation reaction of calcium carbonate in solution. With CO₂ as the foreign substance in solution, the crystal growth method is defined by heterogeneous nucleation, thus, the energetic barrier problems associated with preliminary nucleation are inevitable. Here, carbonic anhydrase reduces the activation energy for the reaction barrier. It is observed from the images that the calcium carbonate crystals are formed immediately under enzymatic catalysis. Crystal growth then results in the gradual expansion of the cross-sectional area of the bridges and the formation of the bridging network between sand particles. In about 20 minutes, most geometrical voids were filled, and the structural system shows strong integrity. To further verify the processes of crystal growth, additional SEM and optical images of the bridging network were performed and are presented in FIG. 15A-FIG. 15C.

Crystal growth in ECM is the diffusion-controlled mechanism. The details of the crystal growth process on the sand-gelatin system are presented in the Crystal bridge growth model, using the diffusion relationships by Fick's First Law. The models were analytically solved to verify the experiment results. The results of the crystal growth modeling framework are presented as the average thickness of the mineral bridges as a function of time in FIG. 8. The theoretical equations are established for crystal growth on the square cross-section beam. The inventors here envision that the calcium carbonate crystals growing on the gelatin of the ECM system is the same as the growth pattern on the polymer. The initial gelatin is considered the polymer beam, and the volume change of the beam is the result of the crystal growth subsequent.

### Example 17: Self-Healing Properties

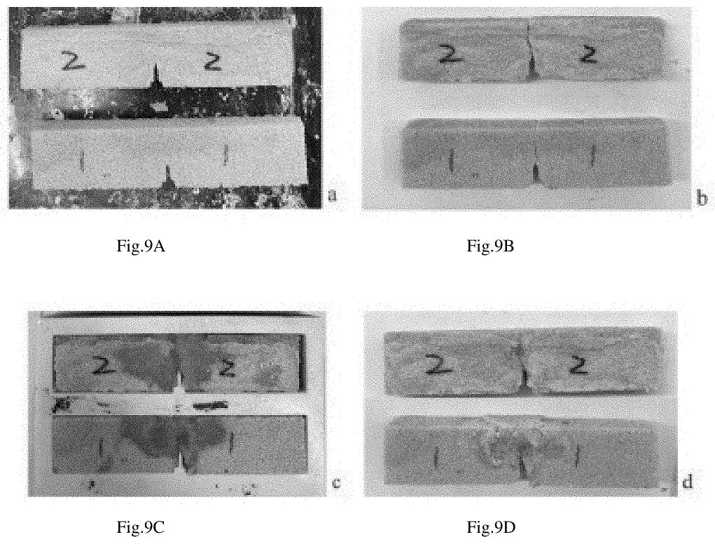

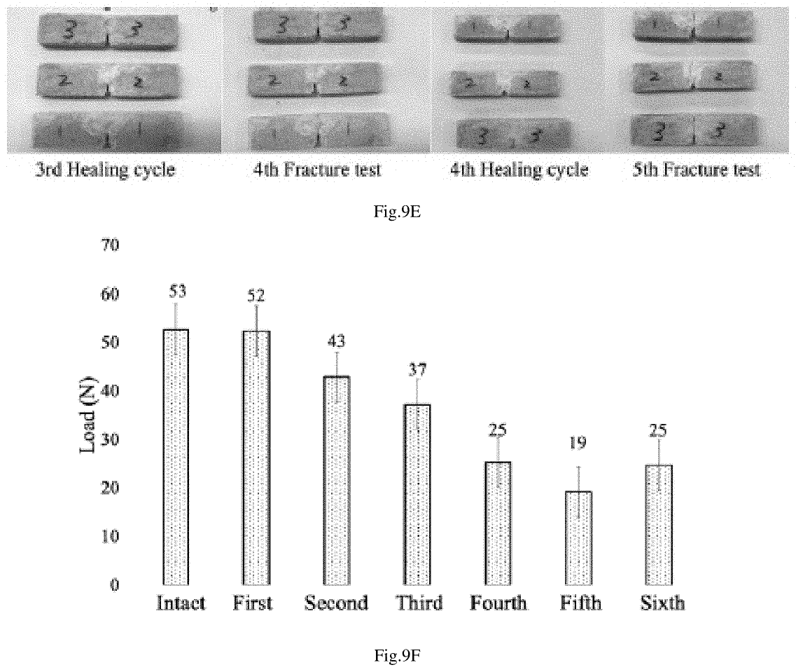

To investigate the self-healing and reconstruction ability of the ECM, it is necessary to simulate the healing process through multiple cycles. For this purpose, an experimental procedure utilizing the three-point bending test was developed. The fracture strength of the beam specimens in each cycle was measured to evaluate the extent of healing in the ECM. In each healing cycle, the fractured samples were placed in a silicon mold and treated with a trace amount of enzyme+calcium solution on the crack surface region (FIG. 9A and FIG. 9B). Pure CO₂ gas was then introduced on the crack surface for 10 minutes. The mineral precipitation occurred predominantly near the crack wake of the specimens. This procedure can be observed as the liquid film on the surface of the samples in FIG. 9C. Healed samples were then fully dehydrated in the oven and demold (FIG. 9D). Three-point bending fracture tests were then again conducted. The prescribed steps were repeated for each cycle and the properties of the healed samples were studied (FIG. 9E). In these figures, the mechanical property of the original sample is termed “Intact,” the first repaired sample is termed “First,” and the second repaired sample is termed “second”, etc.

The results show that the average ultimate loads are similar in intact and first-cycle samples at about 53 N (FIG. 9F). Six samples were tested in each group, and initially, the average maximum load of the second cycle samples decreased to 43 N. This is an 18.8% decrease from the first to the second cycle, a 13.9% decreasing from the second to the third cycle, and 35.1% decrease from the third to the fourth cycle. After the fourth cycle, the ultimate load tends to be stable at about 23 N, which is a reduction from the intact cycle by 52.8%. These results show the ECM beam can reconstruct after six times cycling damage, which shows a self-healing property. These data also support the use of CA and gelatin for the repair to damaged concrete, as shown in further examples herein.

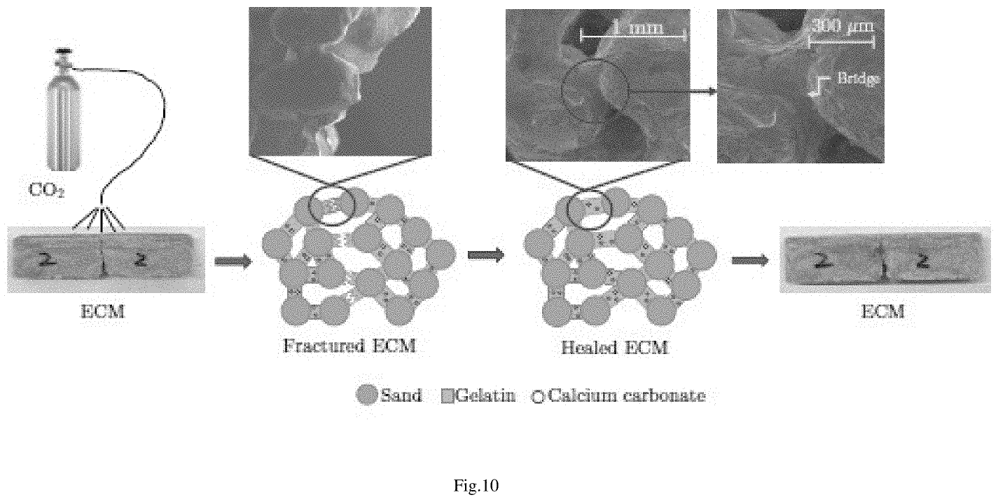

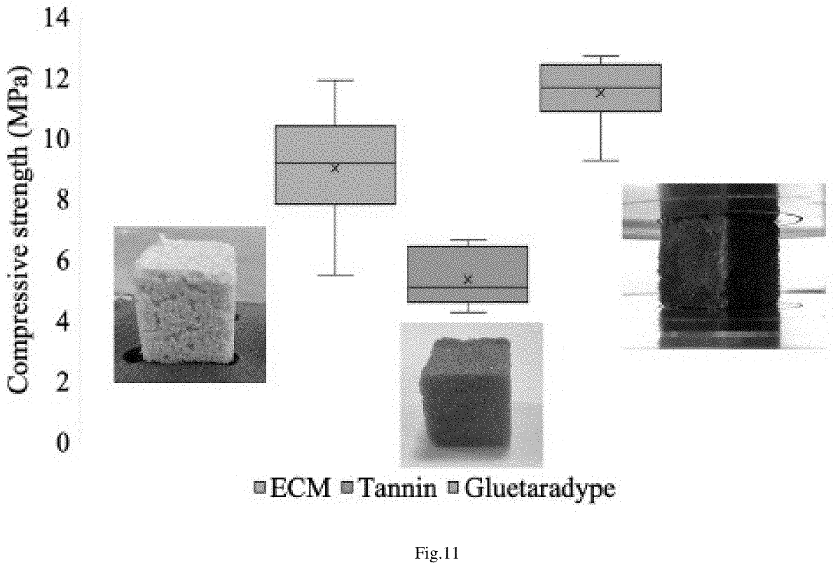

The method demonstrates the damage and healing process of ECM. The entire process only requires a trace amount of calcium source as the filling material. The healing structural materials with a significantly large defect require the addition of some filling materials which is also required in all other self-healing methods such as MICP and Living Building Materials (LBM)s. Furthermore, through the catalysis of carbonic anhydrase, the carbon dioxide in the air can be involved in the self-healing reaction; thus, this process also plays a small role in carbon dioxide sequestration during healing. The results in FIG. 9F show that the average maximum load is decreasing from the second healing cycle with a relatively stable decline up to the fourth cycle. From the fourth cycle of healing, the average maximum load plateaus to about 23 N, which is half of the intact property. Self-healing is the reconstructed structure of crystal gelatin on the substrate perpendicular to crack surfaces that can establish effective crystal bridges as it can be seen in FIG. 10. The SEM analysis of the samples shows that the major fracture mechanism is the failure of mineral bridges. In the self-healing process, the bridges are reestablished after the application of CO₂ (FIG. 10).

It is here envisioned that the entire structural system on the fractured surface was not fully destroyed after the first fracture and the original gelatin-calcium carbonate crystals remained on the surface of the exposed sand grains. From the second healing cycle, the gelatin on the surface of the sand is further dissolved by the aqueous solution, changing the structural integrity at the crack surface. The reason for a stable maximum load after the fourth healing cycle is that the top layer of sand on the fracture surface is exposed without gelatin and those sand particles form bridges to the calcium carbonate crystal directly. This also clearly demonstrates the efficiency of the method. It must be noted that the titration solution will only penetrate from one side to the bottom which leads the gelatin to concentrate in the lower layer, which in turn leads to the creation of a nonuniform crystal bridge structure as the number of healing cycles increases. The data in FIG. 5D and FIG. 5E show that the dehydrated gelatin scaffold significantly contributes to the compressive strength of ECM, so its deterioration weakens the strength of the specimens as the number of healing cycles increases. Moreover, gelatin aging could also occur during multiple healing cycles.

### Example 18: Reinforced ECM

To enhance the physical properties of gelatin, crosslinking agents such as glutaraldehyde, genipin, and microbial transglutaminase are applied. Here, glutaraldehyde was chosen as the ECM crosslinking agent which is by far the most widely used with gelatin, due to its accessibility, low cost, and high efficiency of collagenous material stabilization. Only a very low concentration of glutaraldehyde (1% by gelatin weight) is sufficient to obtain a 100% degree of crosslinking and a 20 times increase in Young's modulus concerning the uncross linked gelatin film. The crosslinking of gelatin with glutaraldehyde involves the reaction of the free amide groups of the lysine or hydroxylysine amino acid residues in the polypeptide chain with the aldehyde group of glutaraldehyde. As the degree of crosslinking increases, the thermal and mechanical properties of gelatin are also increased Tannin is used as an additive to improve the mechanical, thermal, and moisture absorption behavior of gelatin-based adhesives. It was shown that the bond strength (tensile) was increased when tannin is added by 16%. The compressive strength of the results of glutaraldehyde was compared to Tannin in ECM. FIG. 11 displays the compressive strength of glutaraldehyde-modified ECM at around 11.5 MPa, which is 28% higher than the ECM. Meanwhile, tannin modification (27% by gelatin weight) of ECM samples failed to reinforce the ECM structure.

Although glutaraldehyde is an irritant, thousands of successful bio-prosthetic implants have demonstrated that glutaraldehyde cross-linking is clinically acceptable, having reduced cytotoxicity at very low concentrations.

### Example 19: Laser-Induced Curing of ECM-n

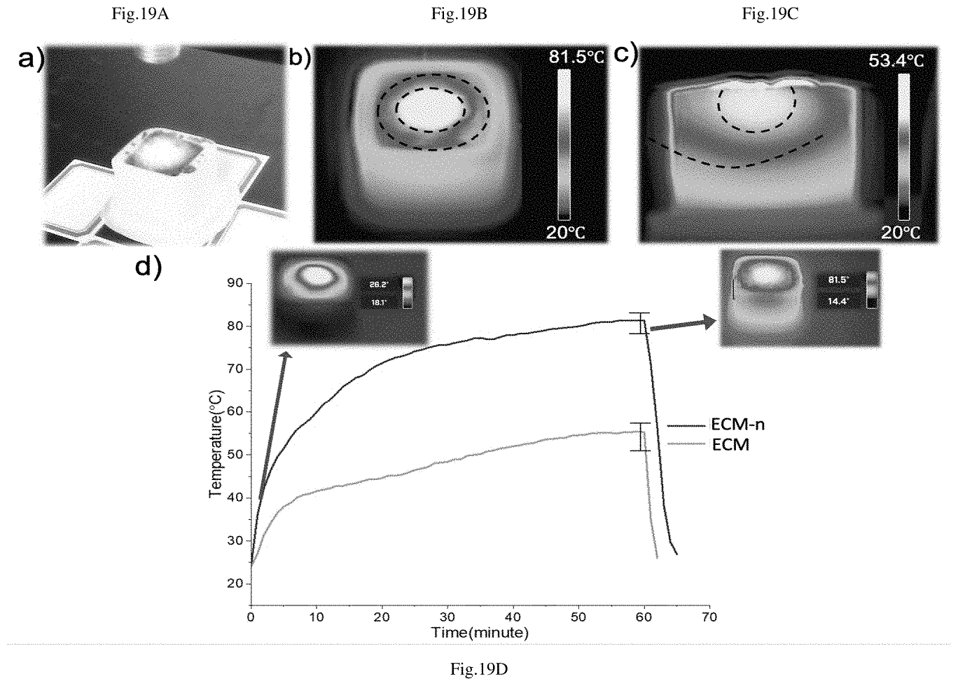

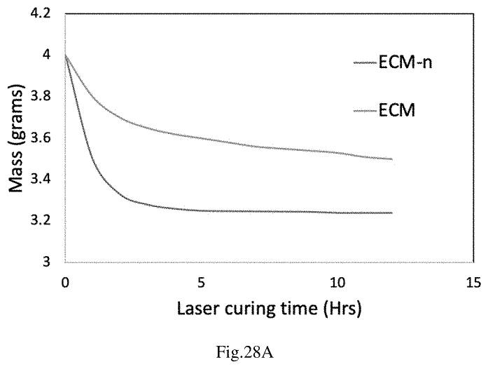

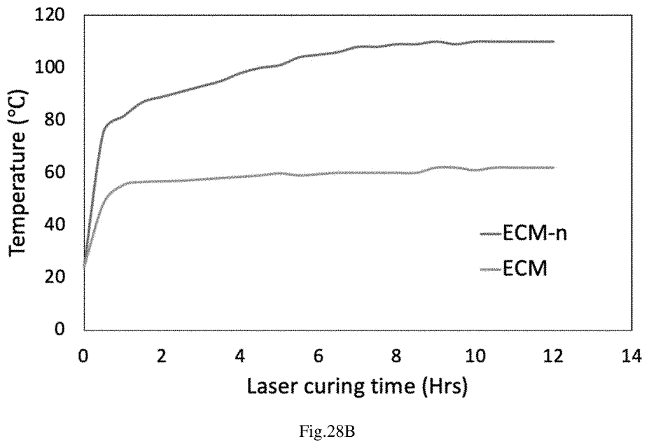

To verify the effect of nanoparticles on ECM curing rate, the 12.5 mm cubic ECM-n (ECM with 0.1% iron oxide nanoparticles) and ECM were fabricated using the same method and subjected to the same power laser. Both specimens were cured under a 3W laser (808 nm wavelength) for 12 hours to obtain a specific degree of dehydration through mass loss evaluation (FIG. 28A). As shown in FIG. 28B, the maximum surface temperature of ECM-n reaches 110° C. in 12 hours to obtain full dehydration, which shows a stable mass after 9 hours. In contrast, the maximum surface temperature of ECM only increases to 62° C. and maintains the mass loss condition. The surface temperature of ECM-n and ECM within the first curing hour was recorded in FIG. 19A-FIG. 19D. FIG. 19A shows the early curing configuration of a cubic 12.5 mm ECM-n in a silicon mold where the sample was placed under a 3W continuous wave laser (808 nm) at a 2 cm distance for 60 minutes, while the surface temperatures at the top and side were recorded by a thermal camera for 70 minutes. It was observed that the maximum surface temperature of the ECM-n sample increases from 26° C. to 81.5° C. (sample: n=5, median: Med=81.5, standard deviation: SD=3), (FIG. 19B and FIG. 19C). In contrast, the temperature of all ECM samples without nanoparticles never rises above 60° C. (n=5, Med=53, SD=5.7). The temperature distribution is non-uniform, with the maximum temperatures occurring within the center of the laser beam. From the temperature curves in FIG. 19D, the heating rate of ECM-n is about twofold higher in the first 20 minutes, the cooling rates of both were similar, reaching room temperature in five and two minutes, respectively, and giving a cooling rate of 11° C./min (n=5, SD=0.82) for ECM-n and 15° C./min for ECM (n=5, SD=1.63). These data indicate that the addition of nanoparticles does not significantly increase the specific heat capacity of ECM. Due to the low specific heat of nanoparticles, the nanoparticles accelerated curing by photothermal effect.

### Example 20: Heat Released Analysis During the Initial Curing

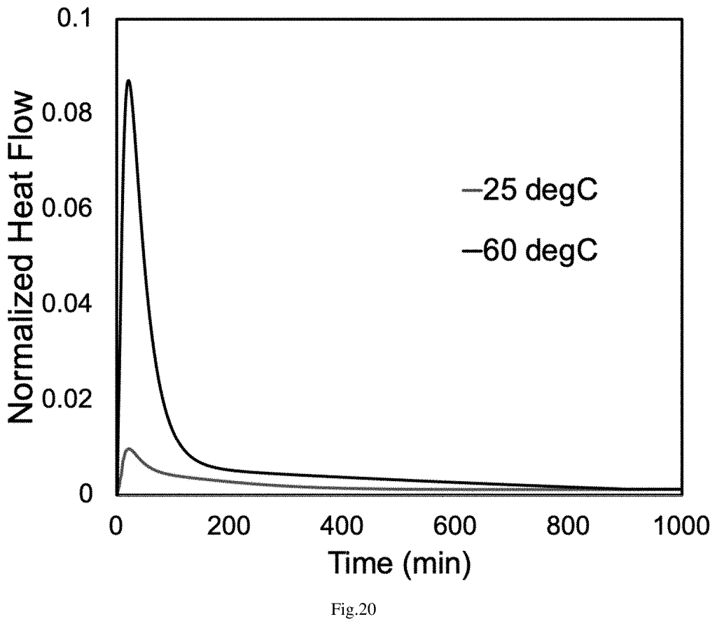

To quantify the early curing kinetics of ECM-n, we used Isothermal calorimetry (ITC) to understand the relationship between the time and heat flow of ECM-n. In FIG. 20, heat flow in ECM-n samples at 25° C. and 60° C. were compared. One heat flow peak was detected at two different temperatures in 30 minutes and both peaks appeared immediately after injecting the enzyme calcite solution, and the peak at 60° C. is more distinct than the peak at 25° C. The intensity of the peak increased with the temperature, indicating that the elevated temperature significantly enhances the degree of dissolution. The higher dissolution of the raw materials at higher temperatures provides more scaffoldings for the following bridging steps, and thus accelerated the early strength development and the final setting of the ECM-n samples.

### Example 21: Laser Heating ECM-n in Low and Room Temperatures

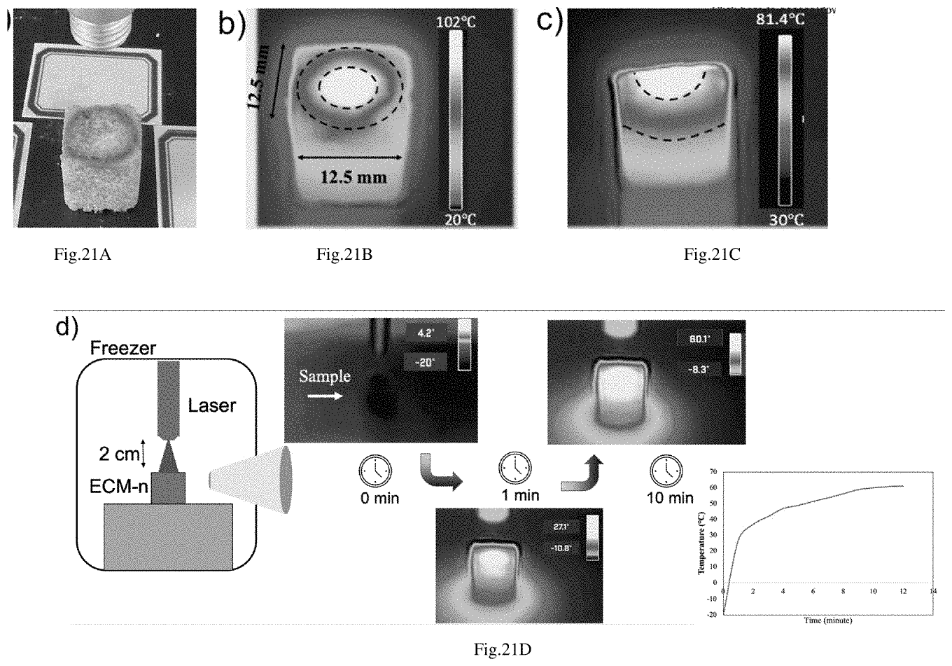

The application of laser heating was also explored on cured ECM-n. FIG. 21A and FIG. 21C demonstrate the photothermal effect on cured ECM-n at ambient temperature induced by a 3W continuous wave laser. FIG. 21A shows the experimental setup. FIG. 21B and FIG. 21C show the top and side views of the contours of temperature for an ECM-n sample subjected to laser heating for about one hour, respectively. For a sample distance of 2 cm from the laser, the peak temperature is concentrated in the center of the sample surface and spreads radially outwards into the surrounding surface region. The results indicated that the maximum temperature of the ECM-n surface increases to 102° C. at room temperature. The influence of heat convection on the side can be observed in FIG. 3C. The temperature profile shows a curve in the form of layers, with the lowest temperature at the furthest position from the heat source.

The photothermal effect of cured ECM-n was also explored at low temperatures. The time sequence of the experiment is presented in FIG. 21D. The sample was stored in a −20° C. freezer to obtain a homogenous temperature on the surface. The schematic on the left shows the experimental setup. The sample was then illuminated in a freezer by the 3W laser. A thermal camera was exploited to record the temperature of the sample surface under illumination for 10 minutes. At the center of the top face of the sample, the temperature increased from −20° C. to a maximum of 60° C. in 10 minutes. The above findings provide a new path for ECM to be utilized as a thermally controllable construction material.

### Example 22: Thermal Modeling of Laser Heating

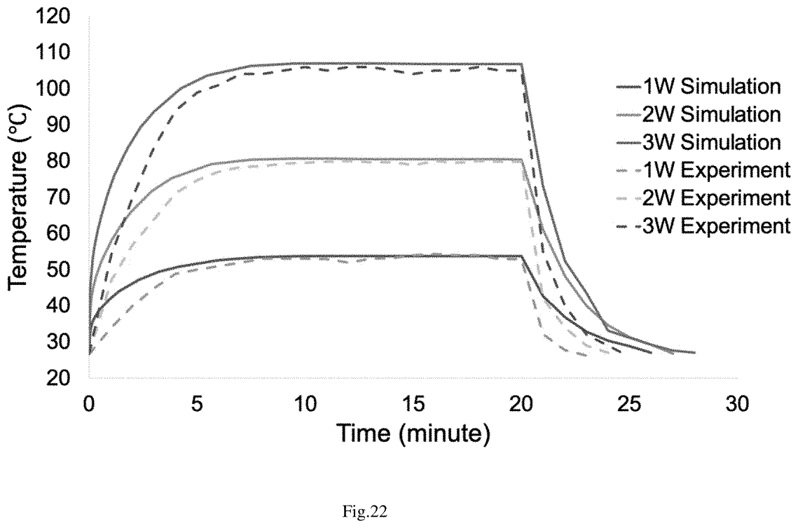

To further demonstrate the thermogenesis ability of the ECM-n, the computational laser heating was modeled using the Finite Element Method (FEM) and the software package COMSOL Multiphysics 6.5 (COMSOL, Inc. Burlington, Mass.). FIG. 22 presents the maximum temperature as a function of time for an ECM-n sample at three different laser powers. It can be observed that the simulation results are slightly less than the corresponding experimental results, and the normalized temperature profiles are proportional to the laser power.

In the process of heating, the ECM-n by laser, heat transfer happens through three main mechanisms: conduction, convection, and radiation. The static laser beam follows Fourier heat conduction law. The heat distribution within the ECM-n is then determined by the following transient heat transfer equation:

$$ρ {C}_{p} \frac{\partial T}{\partial t} = ∇ \cdot ( k ∇ T ) + α Q \qquad ( 15 )$$

where ρ is the density equal to 1660 kg/m³, Cp is the heat capacity of sand equal to 840 J/kg·k, T is the temperature, t is the time, and the thermal conductivity of iron oxide is denoted ask, which is equal to 2.7 W/m·k.

A thermal conductivity parameter was used to represent the iron oxide in this ECM-n model for two reasons: 1) During the dehydration process, the gelatin undergoes a phase change from a gel to a crystalline state, which complicates the modeling process; 2) the iron oxide possesses a higher thermal conductivity than sand and gelatin, which significantly affect the temperature of the system.

The absorption coefficient, a, depends on the object material and the interaction between the object material and the wavelength (808) of the laser, where most models use a constant absorption coefficient by neglecting the influence of incident angle and temperature. To simplify, the absorption coefficient was assumed to be 1, and Q (W/m3) is the laser heat source term. The boundary conditions for Equation (15) at the side of ECM-n are heat transfer by convection, and at the top surface is radiation. The convection (Newton's law of cooling) in the laser heating process is expressed by

q_{con}=h(Tₑₓₜ−T)=−\hat{n}·−(k∇T) (16)

The radiant heat flow rate of the object can be computed based on the empirical formula of Boltzmann's law by

q_{rad}=εσ(T_{amb}⁴−T⁴) (17)

Heat convection always is transferred by gas or liquid media. The air is the major media in the convection, for simplicity, the heat convection coefficient is considered to be uniformly distributed, and equal for all surfaces of the boundary, where, h, the natural air heat convection coefficient is 25 W/m2·k. ε, the emissivity of sand is estimated to be 0.95.[19]σ is the Stefan-Boltzmann constant, and \hat{n} is the direction vector. Text and Tamb are the medium air and room temperatures, respectively, where both are assumed to be 300 K. For a continuous wave laser mode, the fundamental mode of the Gaussian beam is generally preferred and the Gaussian heat source is provided, accordingly. The rate of heat generation by the Gaussian profile of a transverse model optical intensity of order n can be given by

Q(x,y)=pf*f(x,y) (18)

Here, the laser power intensity (W/m2) can be expressed by

$$pf = \frac{P}{π ⋆ {r}^{2}} \qquad ( 19 )$$

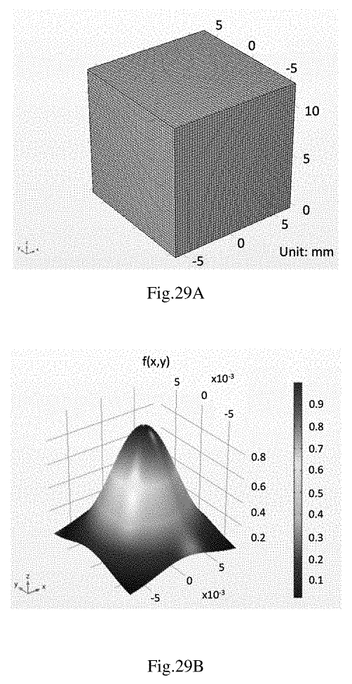

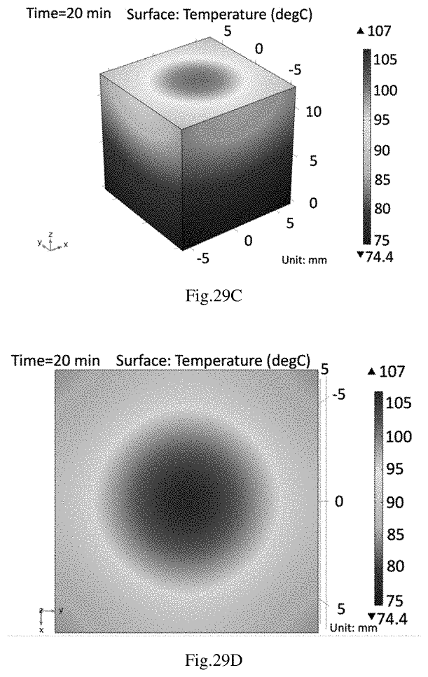

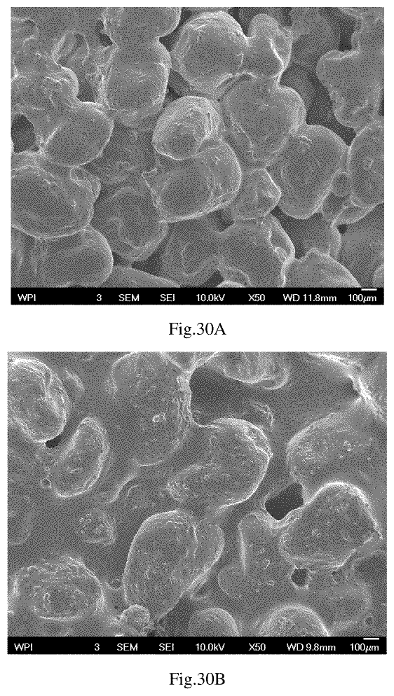

and the Gaussian laser term is

$$f ( x , y ) = exp [ - 2 {( \frac{{x}^{2} + {y}^{2}}{{r}^{2}} )}^{n} ] \qquad ( 20 )$$

where the laser beam was defocused to a processing radius of 6 mm. The Gaussian laser intensity profile of order 1 was implemented in FIG. 29B. The reliability of the simulation was assessed in comparison with the experimental data, according to the heat transfer theory, the temperature stabilized to a relatively steady state at 10 minutes under continuous heat energy input, which is in agreement with the experimental results at different laser power. Both temperature profiles of the experiment and simulation are raised to around 51, 78, and 105° C. at 1, 2, and a 3W laser, respectively.

### Example 23: Self-Healing Assessment Under Laser Curing

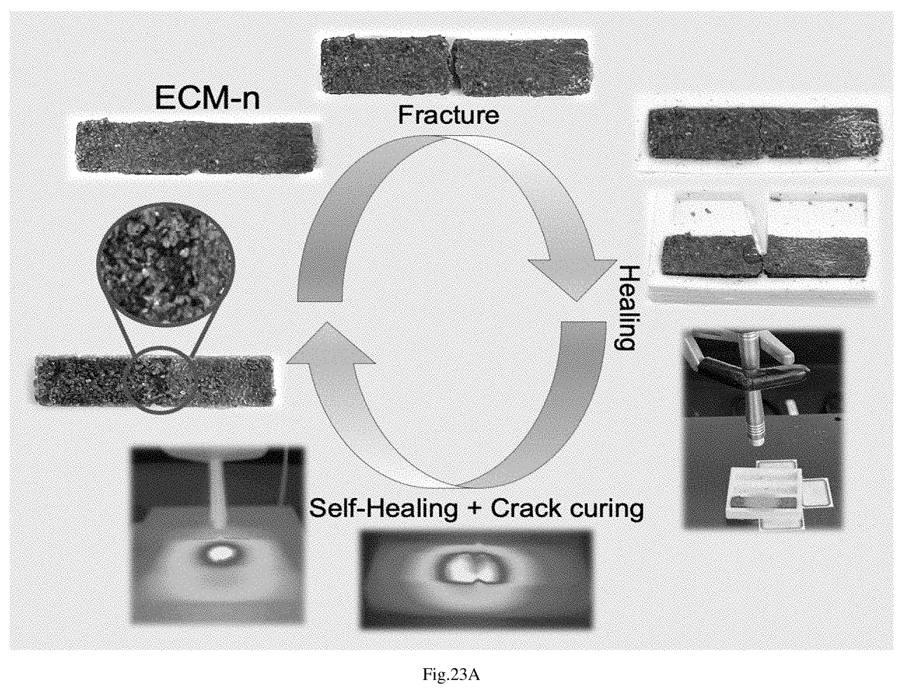

This potential feature was also demonstrated with a similar set of experiments with nanoparticles-modified ECM (ECM-n), which is shown in FIG. 23A-FIG. 23B. It was observed that the ECM-n cuboid sample autonomously self-healed with laser heating without an external force or additional treatment. When fractures occur, the scaffold bridges are exposed on the fracture faces, which implies that the mineral bridges are the weak link in the microstructure of ECM. The fractured sample was assembled in a silicon mold and then the CO₂ gas was sprayed continuously on the fracture region. In the healing process, the precipitated CaCO₃ crystals were expected to gradually grow on the scaffold, and eventually bridge the fracture interface during laser heating. It was noted that the additional carbonic anhydrase was added with the calcium solution in the crack area, this is due to the decreasing enzyme activity during laser photo illumination. The fracture cuboid ECM-n sample was treated with calcium solution with a trace amount of enzyme (5 μL, 2.9 mg/mL) compared with the sample without enzyme treatment in the calcium source agent, which presented more crystals on the repaired crack after laser curing (FIG. 30A-FIG. 30B). These data also provide support for use of carbonic anhydrase and gelatin for the repair of cracked concrete.

The macroscale characterizations of the self-healing process can also be observed from the temperature profile of the sample. The temperature profile contour changes during the healing process, as expected because the nanoparticles are carried by a fluid medium and diffused until the sample is desiccated. The 4.1 mm×3.5 mm fracture surfaces were fully repaired after four hours of laser treatment. The resulting ECM-n has the same self-healing capability as ECM and provides a potential application for outstanding material durability. We also used microcomputed tomography (μ-CT) scanning to characterize and diagnose the internal and external microstructures of cracks within the ECMn matrix (FIG. 23B). The μ-CT scanning images clearly show the crack path disappearing in three display layers (front, center, and back), indicating that the internal crack has self-healed as predicted by laser photo illumination.

### Example 24: ECM-n Morphology and Crystallinity Characterizations

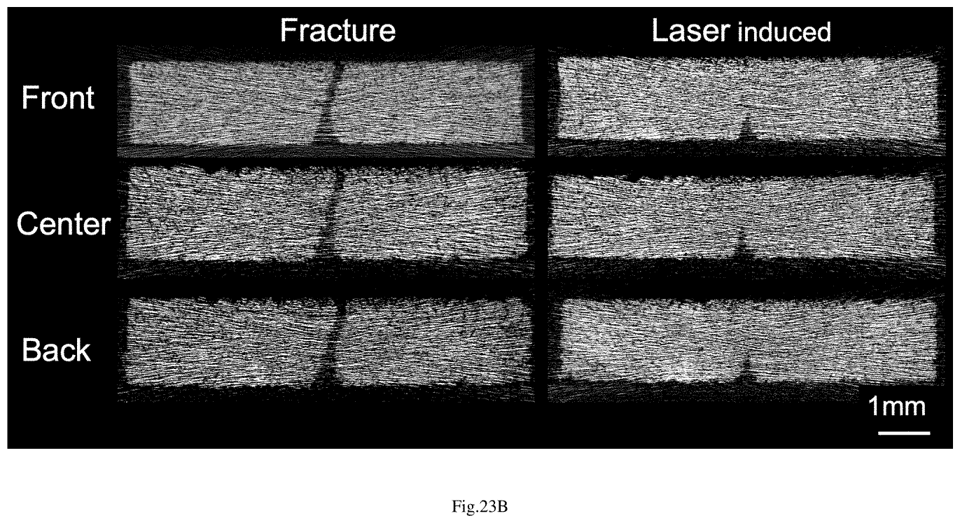

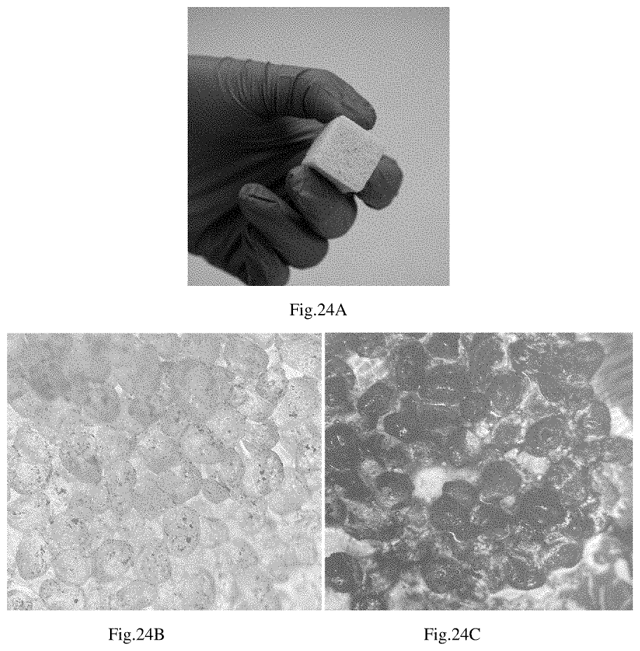

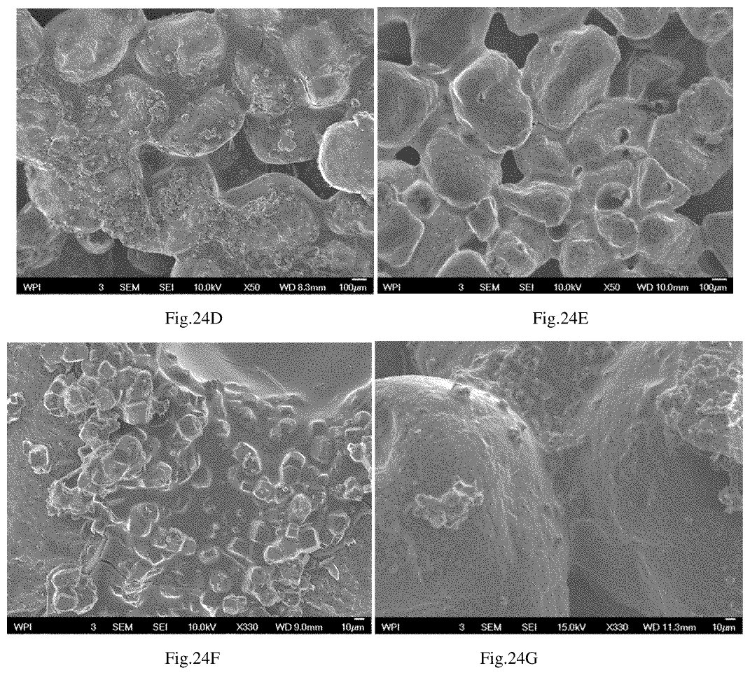

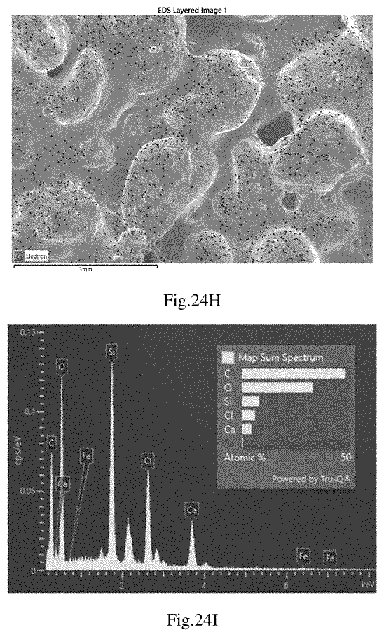

The optical and scanning electron microscope (SEM) images of the morphology of ECM and ECM-n in multiscale were presented in (FIG. 24A-FIG. 24I), showing a variation in calcite crystals size around 10 μm. Detailed morphological characterization indicates that the laser-treated surfaces and the structure of ECM-n are similar to ECM without nanoparticles and cured in an oven. Calcite crystal distribution and the dimension of the hydrogel scaffold were not affected by the laser curing. The ordered structures were well assembled in ECM-n and ECM to efficiently shield the crack propagation. Although the sand particles and calcite crystals were assembled randomly within the polymeric scaffold, the overall view is similar to a body centered cubic (BCC) crystal. In the plane view, the sand particles in both samples are connected with bridges in at least four directions. Due to the characterization of the BCC structural type, sand slurry materials show a high ductility during the bending test, and with a high strain at 0.0032 compared with a normal weight concrete in the range from 0.002 to 0.003. As a result, the local application of the photothermal effect by iron oxide nanoparticles exhibits a mild effect in ECM-n, showing the high extent of stability and mechanical strength in sand slurry materials structure.

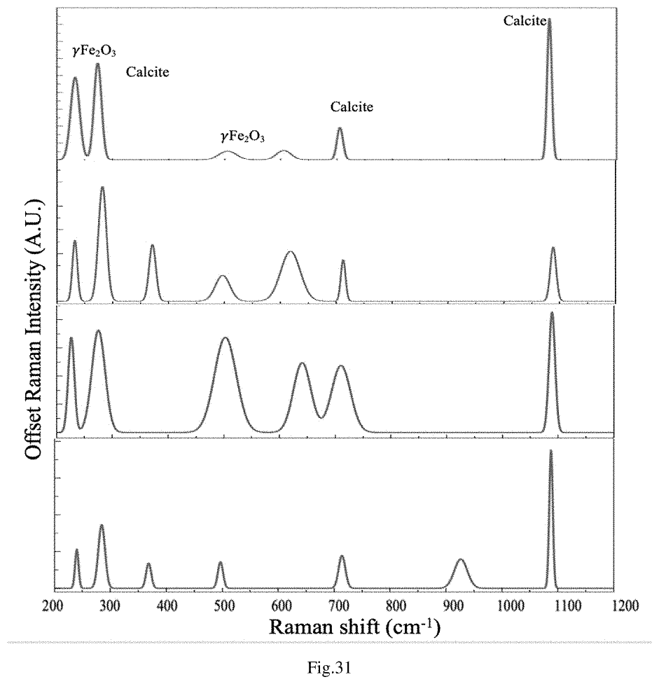

The nanoparticle distribution in ECM-n was also investigated by Energy Dispersion Spectroscopy (EDS), optical microscopy, and Raman spectroscopy, as shown in FIG. 24B, FIG. 24H, and FIG. 31, respectively. The EDS and optical images clearly show the nanoparticles distributed on each of the sand particles, correlating to a spherical temperature profile. The slight aggregation of nanoparticles occurring on the surface is due to the lack of surfactants during nanoparticle preparation. The surfactant coating consists of a variety of ester-based materials that depend on environmental factors at varying levels of chemical complexity, thus, only physical treatment was applied to the nanoparticles by the ultrasonic method to avoid a negative impact on the medium.

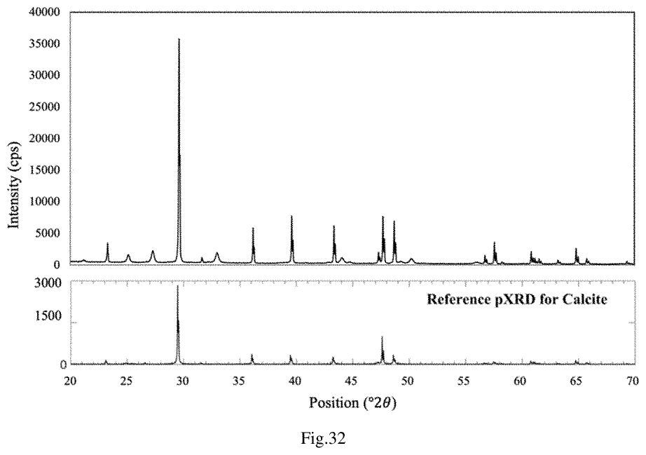

To further identify the distribution of nanoparticles as well as the precipitated crystals, four different locations on the sample surface were spotted by Raman spectroscopy Raman spectroscopy is a nondestructive analytical technique based on changes in the scattering of low-energy light off material and can be used directly on the whole sample. FIG. 31 displays the locations on the ECM-n surface that correspond to calcite and gamma Fe2O3. The spectra were processed and plotted proportionally by fitting the Gaussian function. The Raman characteristic peak of calcium carbonate is relatively narrow, indicating a high extent of crystallization, and the intensity of the peak is higher, indicating a higher content of calcite crystals and iron oxide nanoparticles in this region of the sample surface. The above results show that the nanoparticle distribution in the ECM-n achieves the desired effect. The typical pXRD patterns in FIG. 32 show the presence of precipitated calcium carbonate on ECM-n after laser curing where a diffraction peak is observed at 20=29.6 degrees, which agrees with the reference spectrum for calcite. The result verifies the crystallinity of the enzyme product as calcite. The morphology is supported by the SEM images in FIG. 24A-FIG. 24I.

### Example 25: Mechanical Properties of Repaired ECM-n

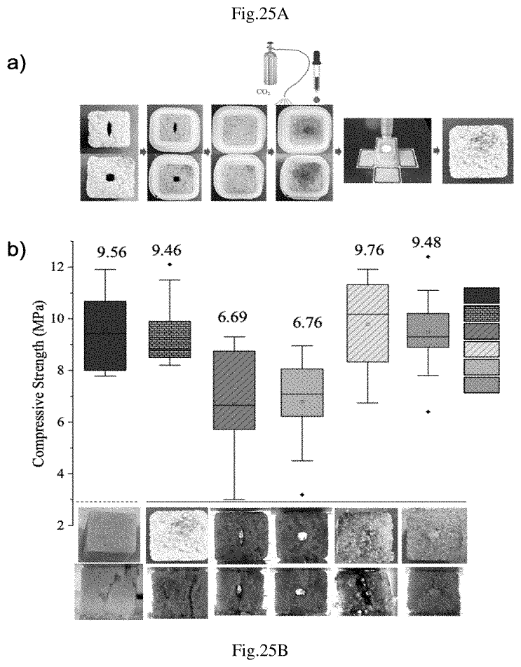

Designing high-performance construction building materials with excellent compressive strength is a long-standing engineering problem. To study the repair performance of the proposed mechanism on laser-induced curing of ECM and ECM-n, cubic-shaped specimens were fabricated for compressive testing, with two types of circular and elliptical flaws. The control and flawed samples are shown in (FIG. 25A-FIG. 25B). It is noted that the ECM-n samples were cured by laser and the flawless ECM samples were cured in the oven. The laser-induced repairing experiment is shown in (FIG. 25A). The normalized values of compressive strengths compared to the control are presented in (FIG. 25B). The ECM-n samples, with a vertically oriented elliptical flaw that is 6 mm tall and 1.5 mm wide and extends through the cubic sample through the entire 12.5 mm depth representing a macroscale crack exhibit about 74% of the strength of the undamaged control specimens. The samples with a circular flaw of 2 mm diameter and extending through the entire depth of the cube sample of 12.5 mm show about 70% of the strength of the undamaged control specimens.

The compressive strength results show that cube-shaped samples with built-in flaws can regain their original compressive strength by adding the calcium enzyme mixed solution to the original sand gelatin matrix. The material was then bubbled with ultra-pure carbon dioxide for ten minutes. This is followed by the application of the laser for curing for 12 hours. The samples with large sizes of the repaired flaws (˜6 mm) exhibit the capability of the proposed method in the repair of the ECM specimens. It is noted that both repaired flaw shape samples slightly outperform the control samples concerning the average compressive strength. The compatibility of the existing matrix with the additional sand-gelatin repair agent is excellent, and the repair process also allows additional curing of the original matrix, leading to an overall stiffer structure.

Comparing the results, the variation in the strength of the elliptical flaw and repaired elliptical flaw ECM-n samples is relatively high. This difference is mainly due to the geometric offset of the elliptical flaw during the sample preparation in the silicone mold. It is noted that the samples with prefabricated elliptical and circular flaws fail by crack initiation at the flaw and growth resulting from dilatation due to Poisson's effect. The p-value of flawed ECM-n samples is less than 0.05, which leads to significant changes in the performance of the structure, while the P-value of repaired elliptical and circular flaw ECM-n are within statistical error (>0.05). Therefore, ECM-n with flaws maintains mechanical stability after repair by the laser.

### Example 26: Embodied Energy and CO₂ of ECM/ECM-n

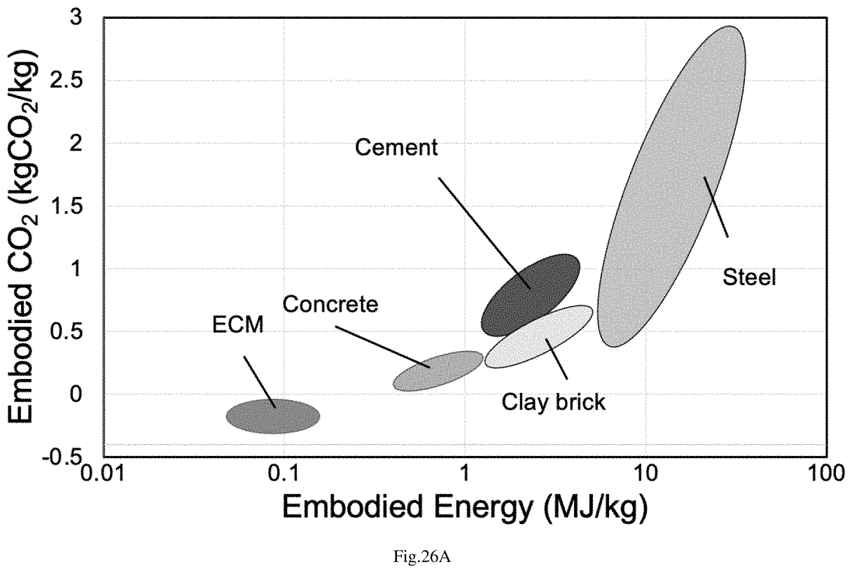

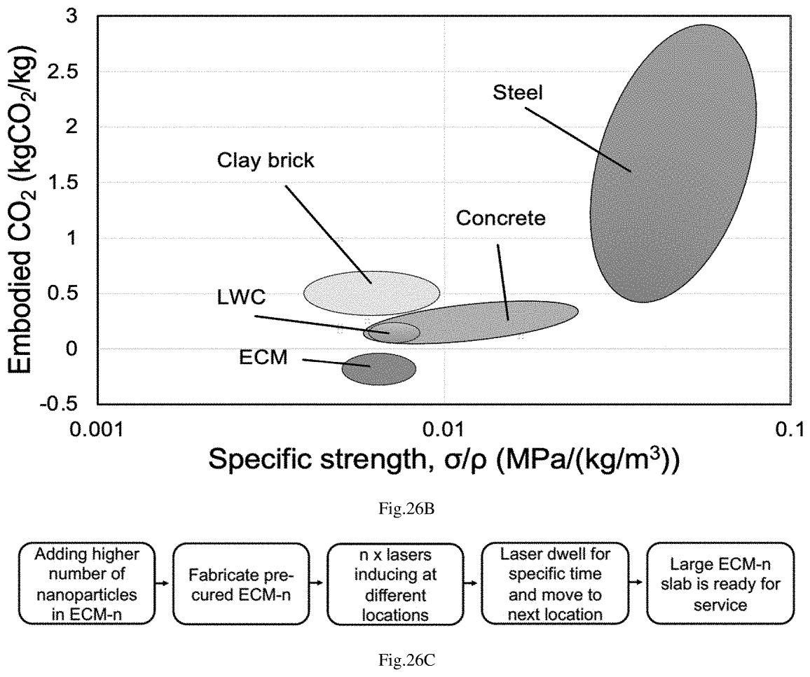

To visualize the impact of ECM/ECM-n on sustainable environmental protection and various types of construction materials, the embodied energy vs. embodied CO₂ and specific strength vs. embodied CO₂ associated with different construction materials were compared (FIG. 26A and FIG. 26B). The Ashby diagram of embodied energy versus embodied strength shows that ECM/ECM-n consumes CO₂ as seen by negative values compared to other construction materials, this is because the formation of the ECM/ECM-n requires the consumption of carbon dioxide. Furthermore, the ECM/ECM-n presents relatively low energy consumption.

The boundaries that were considered in this study are cradle-to-gate. Thus, the transportation-consuming energy and manufacturing processes of ECM/ECM-n were recognized similarly to concrete in this report. The negative CO₂ emissions and low energy embodiment of ECM/ECM-n are because most of the components can be obtained directly in nature or by simple secondary processing. The enzyme, carbonic anhydrase, which is added in trace amounts, is isolated from bovine erythrocytes or other natural sources, which does not generate carbon dioxide, and requires little energy. The calcium can be obtained from natural brines as a by-product of synthetic soda ash production. Another low-carbon method is to produce calcium from hydrochloric acid and limestone. The process will generate hydrogen that can be directly burned or generated by fuel cells to obtain water, which can be achieved by true zero carbon emissions without polluting the environment. The gelatin is extracted by hydrolysis of biological material in a process that is essentially zero-emission. In the methods described herein, only 0.1% (by sand weight) nanoparticles were applied in the medium and the nanoparticles are known to be a clean product that can capture and utilize CO₂ from the air. The specific mechanical properties of ECM/ECM-n were also compared with other construction materials in FIG. 26B. Cracking is assumed to be the initiation of concrete failure. The specific strength of ECM/ECM-n was comparable to the LWC (lightweight concrete) and the minimum of the commercial concrete. There is no negative impact of nanoparticles between ECM and ECM-n, which result in the same mechanical properties.

These materials can entirely replace lightweight concrete and masonry blocks in all their applications. Especially, the ECM-n can rapidly be constructed for the temporary base, secondary structure, and pipe in extreme weather. Here, a guiding flow chart is provided as a reference (FIG. 26C). From the above results, the effective area of ECM-n is proportional to the number of nanoparticles and the power of the laser. Therefore, in on-site construction, a higher number of nanoparticles and higher power of laser are suggested to apply on the ECM-n to guarantee the desired effects. For example: to fabricate a 10 ft×10 ft ECM-n slab, precured ECM-n can be prepared on the foundation, and then ten industrial laser machines can be induced at different spots (assuming the effective area is 0.5 ft×0.5 ft). Designing the laser dwell at each spot for a specific time to achieve full curing (3W laser induces 4 hours for a 12.5 mm depth) before moving to the next spot. The total time should be around 160 hours without considering the ambient humidity and natural drying rate, but this will decrease exponentially with an increase in the number of nanoparticles added. However, achieving the balance between time effectiveness and power cost is the key to this process.

### Example 27: Laser-Induced Curing of ECM-n

The laser diode corresponds to a heating power of 3W. The laser beam was transmitted through a fiber optic cable to the tip of a cylindrical probe before propagating into the sample. The distance between the tip of the probe and the sample surface was set at 2 cm to achieve a laser spot size of 1 cm on the samples. The temperatures of the heated surfaces of samples were recorded with an infrared camera (FLIR One Pro). Subsequently, the 12.5 mm cubic sample was heated and dehydrated in the silicon mold for twelve hours. The cuboid samples (38 mm in length, 8.3 mm in depth, 4.1 mm in width, and 1.1 mm in notch length) were fabricated in the same method. Due to the limited size of the laser beam, the cuboid sample was spotted at three different locations (left, center, and right) for 5 hours at each point to achieve a fully dehydrated specimen.

### Example 28: Laser-Induced Self-Healing of Cuboid Samples

The single-edge notch cuboid sample was fractured into two parts. The parts were assembled and placed into the original mold. 1 mL (5 uL enzyme, concentration: 2.9 mg/mL) of calcium enzyme solution was added to the crack region. Ultra-pure CO₂ was introduced on the surface for fifteen minutes to precipitate the calcite crystals and form a prototype of the bridges. The sample was then settled for another fifteen minutes, allowing the mineralization solution to penetrate naturally into the interior of the structure. The 3W laser was set up 2 cm above the sample and conducted on the crack for 6 hours, then the sample was able to be taken out of the mold.

### Example 29: Repairing Procedure for ECM-n

ECM-n were fabricated and cured in a 3D print silicone mold with an elliptical and circular shape notch. After heating with the 3W laser for 12 hours, the sample was taken out of the mold and transferred to a cubic silicon mold. The notch was filled with well-mixed sand gelatin components, and the sample was then vibrated for thirty seconds. A calcium carbonic anhydrate 200 μL mixed solution with nanoparticles was added to the notched surface and then aerated by CO₂ for 15 minutes. This process should be slow enough to ensure that the solution penetrates to the bottom and does not affect the surrounding structures. 3W laser was induced on the sample from the same distance on the repaired surface for 12 hours and was finally taken out of the mold.

### Example 30: Mineralogical Assessment of Precipitates

The crystal and nanoparticles in ECM-n samples were segregated from the gelatin and sand matrix for assessment of the mineral phase in ECM-n after laser inducing for twelve hours. Dried crystals and nanoparticle mixtures were ground with the mortar and smear-mounted on a sample holder for fingerprint XRD. A Siemens D500 X-ray diffractometer analyzed the samples from 70° 2θ using Cu K-α X-ray radiation with a step size of 0.02° and a dwell time of 2 s per step. Mineral phases were collected using a data collector. The laser-cured ECM-n was evaluated using JEOL JSM-7000F Analytical SEM. Samples were first sputter coated with gold powder. Electron-dispersive spectroscopy (EDS) was employed to assess the nanoparticle distribution in the ECM-n matrix.

### Example 31: Raman Spectroscopy

A hybrid system (XploRA, HORIBA, France) was used to obtain optical images and Raman spectra ranging from 100-1200 cm⁻¹. The experiment was employed with a diode laser (λ=785 nm), and the filter was set up at 10% and ×100 objectives. The acquisition time and accumulation were defined as 5 and 20, respectively.

### Example 32: Heat Released Analysis During the Initial Curing

Isothermal Titration calorimetry (ITC) can measure the thermal power (heat production rate) produced by the hydration reactions of cementitious materials. Additionally, ITC tracks the rate of the overall reaction of the material and visualizes the behavior of the hydration in a way that a simple set time or a compressive strength test does not. The timing and shape of the heat flow curve obtained by calorimetry indicate the relative performance of concrete and potential adverse interactions between materials used in the mixes.

### Example 33: Laser Heating ECM-n In Low and Room Temperatures

All the physical and thermal properties of the ECM-n sample were extracted from experimental data to be used in the FEM. The geometry of the ECM-n was modeled with standard 8-node linear heat transfer elements in three dimensions, which is shown in FIG. 29A. To develop multi-functional properties of ECM, the laser thermal mechanism and heating process in ECM-n were investigated, and simulated the temperature gradient, assuming the material properties are not temperature-dependent since there is no phase change during the heating process. FIG. 29C and FIG. 29D present the highest temperature profile located under the laser beam as a function of time, which agree with the experimental observations in FIG. 21A-FIG. 21C.

### Example 34: ECM-n Morphology and Crystallinity Characterizations

Raman spectroscopy can provide qualitative analysis of sample phases and morphology, crystallinity, and molecular interactions. The peak profiles are generally defined by three parameters: peak intensity, width, and position. The peak intensity is affected by object concentration and orientation. The peak width is related to crystallinity and homogeneity, and the peak position is associated with the components. The carbonate phase (calcite) and gamma Fe2O3 comprise major portions of the material and are relatively easily identified through a phase (˜1086 cm-1) and Fe2O3 band (˜225 cm-1) since both are strong Raman scatterers.

### Example 35: Method and Materials for Repair of Cracks and Fractures

Cracks are assumed as stated above to be the initiating cause of concrete failure. Although very strong, concrete can be brittle and prone to fracture. Current repair methods are inadequate resulting in a weakened material.

The invention described herein provides in one embodiment a method to repair large concrete surfaces using a gelatinous embedded enzyme, carbonic anhydrase, in a calcite solution. This enzyme is found universally in biological systems, for example in humans to regulate the pH of intracellular pools, and fixes carbon dioxide to produce carbonate, an environmentally useful reaction.

The commonly used repair materials for concrete cracks mainly include epoxy systems and acrylic resins. which are all environmentally unfriendly, and often cause delamination or cracking between the original concrete matrix and the repair material. Cracks are the initiators of concrete failure. The present invention provides a method to rapidly repairs larger (<5 mm) cracks in concrete. Repair of small cracks and fractures of small cement samples bathed in a carbonic anhydrase enzyme (CA) has been described (U.S. Pat. No. 10,647,617 issued Feb. 28, 2018). In real world situations where the CA repair solution must be applied to a larger crack on a surface where immersion and deposition are not practical, the repair solution will flow through the material becoming dilute and not accelerating repair. By incorporating CA onto a substrate, such as a silicon bead, or in a solvent that does not allow fast diffusive flow through the concrete, such as hydrogel, or gelatin, an accelerated repair can occur and be maintained.

Presently, calcium nitrate is used as a safe and non-toxic corrosion inhibitor (Al-Amoudi, 0. S. B. et al. Cement and Concrete Composites 2003, 25 (4), 439-449). Treatment of concrete with CA before or after curing not only increases the compressive strength and enables self-healing, and as shown by examples herein, prevents corrosion.

An aspect of the methods herein for concrete repair, corrosion inhibition, and self-healing method is the use of the enzyme carbonic anhydrase (CA).

CA is a small protein that will flow through concrete and not be retained in the structure. Data in Examples herein show that attaching or embedding CA with gelatin better enables repair and self-healing.

Accordingly, the method provided herein in one aspect is used to repair cracks for cementitious surfaces in which the immersion of the surface is not possible or if the deposition of a repair solution is not practical. For example, the concrete surface is vertical, and the repair solution flows through the crack, or the repair solution flows through the material becoming dilute and does not repair. In the methods described herein, a carbonic anhydrase enzyme solution is combined with gelatin for use as a scaffold or the enzyme is incorporated into a substrate such as a silicone bead. In alternative embodiments, the carbonic anhydrase enzyme is dissolved in an aqueous solvent and embedded in a matrix such as a hydrogel, agar, or semi-solid gelatin. Therefore, the enzyme solution when used to apply to a cementitious surface, rather than flow diffusively through the concrete, remains mixed into the cement thereby allowing the repair reaction to continue for the lifetime of the enzyme activity.

In various embodiments of the method, the enzyme used, Recombinant Bos taurus (bovine) CA-II expressed in Escherichia coli, was purchased from Sigma Aldrich. The enzyme was dissolved in 0.1M Tris buffer, 160 mM NaCl, pH 9.5, aliquoted and stored at −20° C. until use. It is envisioned that one or more carbonic anhydrase enzymes from any source would be appropriate, for example, obtained from bovine, porcine or ovine waste products such as blood or offal. Bacterial and yeast enzymes are likewise suitable, obtained from cellular mat material byproducts of high-value protein fermentations or other industrial sources. It is envisioned that carbonic anhydrases obtained from a thermophilic species may be advantageous for use in cement repair in a tropical climate, and similarly from a xerophilic bacterium advantageous in a cold climate.

Enzyme suitable to the methods herein need not be purified nor recombinantly produced, for example, crude cells extracts precipitated with ammonium sulfate to 60% to 65% saturation, or extracted with organic solvents and redissolved may be sufficient for large-scale industrial concrete use. Methods of purification of carbonic anhydrase are well known, see for example, da Costa et al., Chemosphere 88 (2012) 255-259.

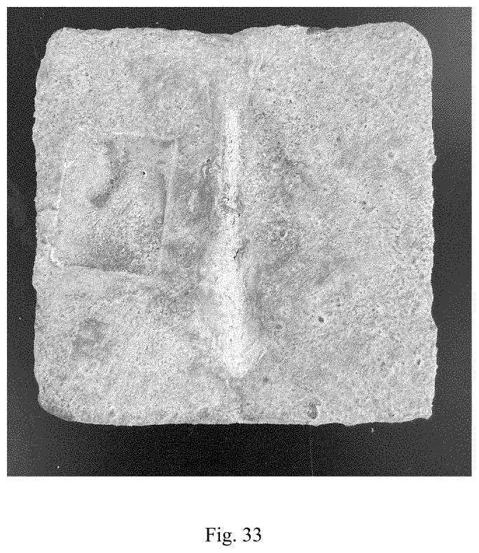

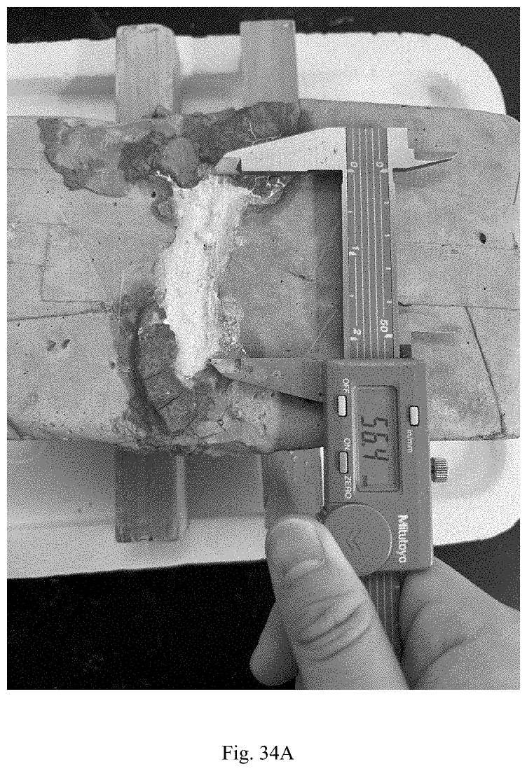

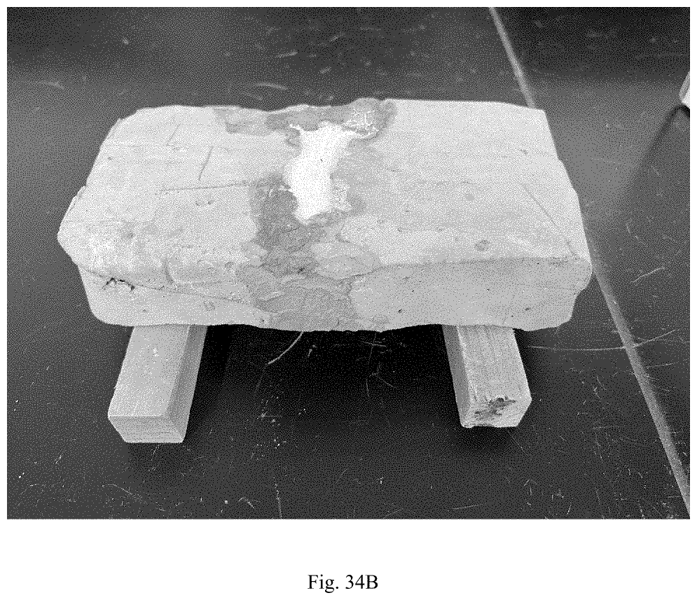

In the Examples shown in FIGS. 33 and 34A and 34B herein the recombinant bovine enzyme product was precipitated from a 49.6:49.6:0.74:0.0001 volume percent solution of four parts in a beaker on a stir plate: 200 mL of 2M calcium chloride dihydrate, 200 mL of 0.1M Tris base, ultrapure water, and a 5 μL aliquot of 20 mM bovine CA supplied in 150 mM sodium chloride. The amount of carbonate anhydrase enzyme (CA) used was very small, and being a catalyst is recycled through multiple rounds of production. The primary 50:50 percent solution of 2M CaCl₂ and 0.1M Tris base had an initial pH of 9.5. The pH was observed to have been reduced as a result of enzyme activity by 0.1 units upon the addition of bubbled CO₂ gas. The 5 μL aliquot of the CA solution was rinsed from its container with 3 mL of ultrapure water and added to the beaker. The CO₂ gas was continuously bubbled for 20 minutes and the pH of the solution was observed to have reduced from 9.5 to 7.4.

The CA solution was left to stand for 24 hours and then the supernatant was removed. The CA solution was stirred well for two min and 20 mL of the solution was siphoned and heated to 35° C. Gelatin (Knox, 1 g) was mixed with the 20 mL solution for two minutes, stirring the solution continuously until the gelatin was dissolved in the resulting 5% gelatin solution. The concrete slab was placed in two stands so that the notch was suspended. A volume of 10 mL of the CA solution with gelatin was filled in the notch evenly and the notch was entirely covered by the solution. The concrete slab and the notch were airdried for 24 hours. CA solution with gelatin 10 mL was again applied to the notch. After 24 hours of air drying, 7 mL of CA solution without gelatin was filled in the notch. The slab and the notch were allowed to air dry for 4 hours, and the notch was again filled with 7 mL of the CA solution without gelatin. The procedure of air-drying for 4 hours and reapplying the CA solution without gelatin was repeated for 5 days. The CA solution with gelatin maintained the seal and the CA solution without gelatin stayed in the notch. After thoroughly airdrying, the notches of various sizes were observed to have been repaired after 7 days and after 14 days, respectively, as shown in FIGS. 33, 34A, and 3B.

Further provided herein are methods for prevention of corrosion of concrete and materials including a concrete corrosion inhibitor admixture using carbonic anhydrase enzyme to mitigate the chloride permeability in concrete, as demonstrated by data shown in the Figures herein. A major factor in extending the life of the concrete structure is the use of corrosion inhibitors. The Global Corrosion Inhibitor Market is projected to grow at a CAGR of 3.54% from USD 7.70 Billion in 2020 to USD 10.09 Billion in 2028. The enzyme-based corrosion inhibitor provided herein improves the durability of concrete structures by significantly reducing the porosity of the concrete during curing.

The reduced porosity of the resulting concrete extends the life of the concrete structures by blocking the access of harmful ions such as chloride. Chloride is deleterious to concrete, and is a major factor in concrete failure in coastal regions. Porosity is assayed by the physical analytical methods shown in Figures herein. In actual use during construction, porosity can be determined more quickly and conveniently on site also by use of visible dyes to assess reduced porosity and consequential increased corrosion resistance. Negatively charged dyes that mimic chloride ions in size and charge are preferably used. Coomassie Blue or brilliant blue which has a negative charge at neutral pH is an exemplary dye for this purpose. See also Savicheva, E. et al. Angewandt. Chem. Int. Ed. Engl (2020) 59 (14): 5505-5509.

Globally, concrete is a trillion-dollar industry. The inexpensive additive provided herein, as a corrosion inhibitor, greatly extends the life of concrete structures in buildings, roads, pavements, etc. All current corrosion inhibitors negatively affect the plasticity and strength of concrete. Three sets of experimental results independently show the superior performance of the enzymatic corrosion inhibitor methods and materials herein, in comparison to all current corrosion inhibitors in the market, while having no negative effects on the mechanical properties of concrete.

Almost all current corrosion inhibitors in the market are calcium nitride-based. While calcium nitride can be used as a corrosion inhibitor to reduce concrete porosity, it has negative effects on the mechanical properties of concrete such as strength. Data herein show that the enzyme-based method does not have any harmful effect on concrete.

Almost all current corrosion inhibitors in the market are calcium nitride-based. While calcium nitride can be used as a corrosion inhibitor to reduce concrete porosity, it has negative effects on the mechanical properties of concrete such as strength. The enzyme-based method provided herein does not have any harmful effect on concrete, and in contrast, enzyme-based methods and materials are self-healing.

Various configurations depicting the above features and benefits as disclosed herein are shown and described further in claims and examples. The contents of all cited references are hereby incorporated herein in their entireties.

What is claimed is:

1. A method for repairing at least one fracture or notch in a cementitious surface, the method comprising:
treating the at least one fracture or notch in the cementitious surface to at least one carbonic anhydrase preparation, the preparation containing carbonic anhydrase immobilized non-covalently on a particulate substrate or embedded in a semi-solid bead matrix; and
subjecting the cementitious treated surface to ambient conditions of atmospheric carbon dioxide and temperature, thereby repairing the at least one fracture.

2. The method according to claim 1 further comprising repeating the treating the at least one fracture or notch with the carbonic anhydrase preparation and subjecting the treated surface to the ambient condition.

3. The method according to claim 2, further comprising contacting the at least one carbonic anhydrase preparation, air drying the treated cementitious surface before repeating the treating.

4. The method according to claim 1, the carbonic anhydrase preparation further comprises at least one component selected from: a native carbonic anhydrase, calcium ions, a buffer, and water.

5. The method according to claim 1, the particulate substrate of the carbonic anhydrase preparation is at least one selected from: a silicone bead, a silica gel, silicon dioxide, disiloxane, silicic acid, silanol, an organic silicon compound, sand, grit, cellulose and cellulose derivatives.

6. The method according to claim 1, the carbonic anhydrase preparation semi-solid bead matrix is prepared from at least one selected from: a hydrogel, an agar, and a gelatin.

7. The method according to claim 1, the carbonic anhydrase is recombinantly-produced.

8. The method according to claim 1, the carbonic anhydrase is purified or partially purified from a biological source, for example, is a mammalian enzyme byproduct of a meat industry produced for example from bovine blood or is a bacterial enzyme or yeast enzyme byproduct of antibiotic or high-value protein fermentation.

9. The method according to claim 8, the bacterial enzyme is from a thermophilic or a xerophilic species.

10. A method for improving durability and corrosion resistance of a cementitious surface, the method comprising:
contacting a cement mixture prior to curing to at least one carbonic anhydrase (CA) preparation to form an enzyme-cement mixture (ECM);
applying the ECM to a surface and subjecting the mixture on the surface to an ambient atmosphere and curing, removing core cylindrical samples and observing increased crystallization or decreased uptake of a visible dye, preferably the dye is negatively charge as to mimic chloride ions, in the samples after a time period of at least two days to at least seven days, thereby reducing porosity and conferring corrosion resistance in a resulting CA-contacted cementitious surface in comparison to a control cement not CA-contacted.

11. The method according to claim 10, the carbonic anhydrase preparation comprises CA immobilized non-covalently on a particulate substrate selected from a silicone bead, a silica gel, sand, grit, cellulose and cellulose derivatives; or the carbonic anhydrase preparation comprises CA embedded in a semi-solid bead matrix comprising hydrogel or in gelatin.

12. The method according to claim 10, reducing porosity of the resulting CA-contacted cementitious surface further comprising reducing permeability of the surface to deleterious environmental corrosive agents.

13. The method according to claim 10, reducing porosity of the resulting cementitious surface comprises reducing permeation of deleterious salt ions including chloride salts.

14. The method according to claim 10, the CA is selected from at least one of:
recombinantly produced; purified from a food industry byproduct; partially purified from a food industry byproduct; purified from a fermentation byproduct; and, partially purified from a fermentation byproduct.

15. The method according to claim 10, wherein applying the ECM further comprises adding Ca⁺⁺ ions to the surface.

16. The method according to claim 15, the Ca⁺⁺ is calcium formate.

17. The method according to claim 10, wherein contacting the cement further comprises adding Ca⁺⁺ ions to the mixture.

18. A corrosion-resistant cement mix having improved durability, the cement mix comprising carbonic anhydrase enzyme in an aqueous solution in a particulate admixture, the enzyme associated with at least one selected from silicone beads, silica gel, sand, grit, cellulose and cellulose derivatives, at a pH of 4.5-9.5.

19. The corrosion-resistant cement mix according to claim 18, the enzyme present at a concentration in a range selected from the group of: 100 nM to 500 nM, 500 nM to 1 μM, 1 μM to 5 μM, and 5 μM to 10 μM.

20. The corrosion-resistant cement mix according to claim 18, further comprising Ca⁺⁺.
